(19) United States
(12) Patent Application Publication
(10) Pub. No.: US 2025/0043256 A1
(43) Pub. Date: Feb. 6, 2025

(54) VARIANTS OF THERMOSTABLE DNA PRIMASES AND USES THEREOF

(71) Applicant: Univercells France SAS, Evry-Courcouronnes (FR)

(72) Inventors: Irina RANDRIANJATOVO-GBALOU, Evry-Courcouronnes (FR); Ahmed SAID, Evry-Courcouronnes (FR); Renaud RAHIER, Evry-Courcouronnes (FR)

(21) Appl. No.: 18/744,396

(22) Filed: Jun. 14, 2024

Related U.S. Application Data
(63) Continuation of application No. PCT/EP2022/086151, filed on Dec. 15, 2022.

(30) Foreign Application Priority Data
Dec. 15, 2021 (EP) EP21306786.1

(51) Int. Cl. C12N 9/12; C12P 19/34

(52) U.S. Cl. CPC C12N 9/1264; C12P 19/34; C12Y 207/07031

(57) ABSTRACT

The present invention relates to a functionally active mutated primase domain from an archaeal DNA primase belonging to the primase-polymerase family, comprising at least one amino acid substitution, wherein said mutated primase domain retains at least 50% of the template-independent terminal nucleotidyl transferase activity of the corresponding wild-type primase domain.

### CROSS REFERENCE

This application is a continuation of International Application No. PCT/EP2022/086151, filed Dec. 15, 2022, which claims priority to EP Application No. 21306786.1, filed Dec. 15, 2021, all of which are incorporated herein by reference in their entireties.

### FIELD OF INVENTION

The present invention relates to the field of nucleic acid synthesis or sequencing. In particular, it relates to functionally active mutated primase domain from an archaeal DNA primase belonging to the primase-polymerase family, comprising at least one amino acid substitution, wherein said mutated primase domain retains at least 50% of the template-independent terminal nucleotidyl transferase activity of the corresponding wild-type primase domain.

### REFERENCE TO A SEQUENCE LISTING

The instant application contains a Sequence Listing which has been submitted in XML file format and is hereby incorporated by reference in its entirety. Said XML copy, created on Oct. 2, 2024, is named 60903-728_301_Replacement_SL.xml and is 52,572 bytes in size.

### BACKGROUND OF INVENTION

Template-independent, sequence-controlled synthesis of nucleic acids represents a major industrial challenge.

Many industries are capable of synthesizing DNA or RNA strands by chemical means; however, they have quickly experienced the limits of these manufacturing processes. Today, the gold method for chemical synthesis of nucleic acids is the phosphoramidite method, developed in the 1980's, and later enhanced with solid-phase supports and automation (Beaucage & Caruthers, 1981. Tetrahedron Lett. 22(20):1859-62; McBride & Caruthers, 1983. Tetrahedron Lett. 24(3):245-8; Beaucage & Iyer, 1992. Tetrahedron. 48(12):2223-2311).

However, this method shows major limitations: first, nucleic acids with no more than around 250 nucleotides can be synthetized. Second, the phosphoramidite method requires the use of organic solvents which can be carcinogens, reproductive hazards, and neurotoxins; while synthetic byproducts can further be toxic and polluting.

In order to overcome these problems, a method of template-independent, sequence-controlled nucleic acid synthesis by enzymatic means has recently been developed, based on the use of a terminal deoxynucleotidyl transferase (TdT), an enzyme which is able to polymerize single-stranded DNA fragments in the absence of template strand. This “template-independent” property was hence exploited for the sequence-controlled synthesis of nucleic acids, using reversibly 3′-blocked nucleoside triphosphates.

However, the use of TdT also has its own limits, in particular during the polymerization of long nucleic acids, or of sequences rich in guanine-cytosine. Indeed, in these two cases, the synthetized DNA strand tends to fold in on itself and to form secondary structures, thereby masking the 3′ position of the strand and ultimately leading to a drastic reduction in the final synthesis yield.

Methods are being explored to work around this problem. In particular, authors in Singapore have recently developed an assay to identify thermostable TdT variants (Chua et al., 2020. ACS Synth Biol. 9(7):1725-1735). In brief, they generated a library of TdT mutants using an error-prone polymerase, and screened about 10000 of these TdT mutants. They finally identified one TdT variant that was 10° C. more thermostable than the wildtype TdT of bovine origin (which optimum temperature is around 37° C., with an unfolding Tm around 40° C.), while preserving its catalytic properties. In the same time, another research group has reported a similar outcome using an in silico-driven approach to identify a TdT variant that was 10° C. more thermostable than the wildtype TdT from Mus musculus (Barthel et al., 2020. Genes (Basel). 11(1):102).

Although promising, this finding did not resolve all the issues explained above.

Recently, the Inventors have identified several archaeal DNA primase domains that are capable of template-independent terminal nucleotidyl transferase activity, at temperatures between 60° C. and 95° C. (International patent applications WO 2021/250269 and WO 2021/250265).

They now describe mutants of these archaeal DNA primase domains, which retain their template-independent terminal nucleotidyl transferase activity, or even exhibit improved activities compared to the wild-type domains previously described.

### SUMMARY

The present invention relates to a functionally active mutated primase domain from an archaeal DNA primase belonging to the primase-polymerase family, comprising at least one amino acid substitution, wherein said mutated primase domain retains at least 50% of the template-independent terminal nucleotidyl transferase activity of the corresponding wild-type primase domain.

In one embodiment, the mutated primase domain has at least an equivalent template-independent terminal nucleotidyl transferase activity compared to the corresponding wild-type primase domain.

In one embodiment, the mutated primase domain has an improved template-independent terminal nucleotidyl transferase activity compared to the corresponding wild-type primase domain.

In one embodiment, the at least one amino acid substitution is at a position positionally equivalent to N217, K234, N206, L229, Y233, K236, T230, Y122, F74, F174, F219, 1231, P238, Y235, P228, S68, and/or N232 in the primase domain of the wild-type Pyrococcus sp. 12-1 DNA primase with SEQ ID NO: 2.

In one embodiment, the at least one amino acid substitution is at a position positionally equivalent to N217, K234, N206, L229, Y233, K236, T230, Y122, F74, F174, F219, 1231, and/or P238 in the primase domain of the wild-type Pyrococcus sp. 12-1 DNA primase with SEQ ID NO: 2.

In one embodiment, the at least one amino acid substitution is at least one amino acid substitution is at a position positionally equivalent to N217, K234, N206, L229, Y233, K236, T230, Y122, and/or F74 in the primase domain of the wild-type Pyrococcus sp. 12-1 DNA primase with SEQ ID NO: 2.

In one embodiment, the at least one amino acid substitution comprises:
a substitution of the positionally equivalent N217 with an amino acid residue comprising a positively charged side chain such as a lysine (K), arginine (R), or histidine (H) residue; preferably with a lysine (K), or arginine (R) residue; more preferably with a lysine (K) residue;
a substitution of the positionally equivalent K234 with an amino acid residue comprising a positively charged side chain such as an arginine (R), or histidine (H) residue; preferably with an arginine (R) residue;
a substitution of the positionally equivalent N206 with an amino acid residue comprising a positively charged side chain such as an arginine (R), histidine (H), or lysine (K) residue; preferably with an arginine (R) residue;
a substitution of the positionally equivalent L229 with an amino acid residue comprising an amine-containing side chain, such as an asparagine (N), arginine (R), glutamine (Q), or lysine (K) residue, or a hydrophobic side chain such as an alanine (A), glycine (G), valine (V), isoleucine (I), or methionine (M) residue; preferably with an asparagine (N), alanine (A), glycine (G), or arginine (R) residue; more preferably with an asparagine (N) or alanine (A) residue;
a substitution of the positionally equivalent Y233 with an amino acid residue comprising a positively charged side chain such as a lysine (K), histidine (H) or arginine (R) residue, or a hydrophobic side chain such as an alanine (A), glycine (G), valine (V), isoleucine (I), leucine (L), or methionine (M) residue; preferably with a lysine (K), histidine (H), arginine (R), or alanine (A) residue; more preferably with a lysine (K) or histidine (H) residue;
a substitution of the positionally equivalent K236 with an amino acid residue comprising a positively charged side chain such as an arginine (R), or histidine (H) residue; preferably with an arginine (R) residue;
a substitution of the positionally equivalent T230 with a cysteine (C) residue, or an amino acid residue comprising a hydrophobic side chain such as an alanine (A), glycine (G), valine (V), isoleucine (I), leucine (L), or methionine (M) residue, or a polar uncharged side chain, such as a serine (S), asparagine (N), glutamine (Q), or threonine (T) residue; preferably with a cysteine (C), alanine (A) or serine (S) residue; more preferably with a cysteine (C) residue;
a substitution of the positionally equivalent Y122 with an amino acid residue comprising an aromatic side chain, such as a histidine (H), phenylalanine (F), or tryptophan (W) residue, or a hydrophobic side chain such as an alanine (A), glycine (G), valine (V), isoleucine (I), leucine (L), or methionine (M) residue; preferably with an alanine (A), or histidine (H) residue; more preferably with a histidine (H) residue;
a substitution of the positionally equivalent F74 with an amino acid residue comprising an aromatic side chain, such as a tyrosine (Y), tryptophane (W), or histidine (H) residue, or an amine-containing side chain, such as a glutamine (Q), asparagine (N), arginine (R) or lysine (K) residue; preferably with a tyrosine (Y), or glutamine (Q) residue; more preferably with a tyrosine (Y) residue;
a substitution of the positionally equivalent F174 with an amino acid residue comprising an amine-containing side chain, such as an arginine (R), glutamine (Q), asparagine (N), or lysine (K) residue, or an amino acid residue comprising an aromatic side chain, such as a tyrosine (Y), tryptophane (W), or histidine (H) residue; preferably with an arginine (R) residue;
a substitution of the positionally equivalent F219 with an amino acid residue comprising an aromatic side chain, such as a tyrosine (Y), histidine (H), or tryptophan (W) residue; preferably with a tyrosine (Y) residue;
a substitution of the positionally equivalent 1231 with an amino acid residue comprising a hydrophobic side chain such as an alanine (A), glycine (G), valine (V), leucine (L), or methionine (M) residue, or a positively charged side chain such as a lysine (K), arginine (R), or histidine (H) residue; preferably with an alanine (A), lysine (K) or arginine (R) residue;
a substitution of the positionally equivalent P238 with an amino acid residue comprising a positively charged side chain such as an arginine (R), histidine (H), or lysine (K) residue; preferably with an arginine (R) residue;
a substitution of the positionally equivalent Y235 with an amino acid residue comprising an aromatic side chain, such as a phenylalanine (F), tryptophan (W), or histidine (H) residue; preferably with a phenylalanine (F) or tryptophan (W) residue; more preferably with a phenylalanine (F) residue;
a substitution of the positionally equivalent P228 with an amino acid residue comprising a hydrophobic side chain, such as an alanine (A), glycine (G), valine (V), isoleucine (I), leucine (L), or methionine (M) residue, or a polar uncharged side chain, such as an asparagine (N), glutamine (Q), threonine (T) or serine (S) residue; preferably with an alanine (A) or asparagine (N) residue;
a substitution of the positionally equivalent S68 with an amino acid residue comprising a polar uncharged side chain, such as an asparagine (N), glutamine (Q), or threonine (T) residue; preferably with an asparagine (N) residue; and/or
a substitution of the positionally equivalent N232 with an amino acid residue comprising an amine-containing side chain, such as an arginine (R), asparagine (N), glutamine (Q), lysine (K) or histidine (H) residue; preferably with an arginine (R) residue;
   in the primase domain of the wild-type Pyrococcus sp. 12-1 DNA primase with SEQ ID NO: 2.

In one embodiment, the at least one amino acid substitution is positionally equivalent to N217K, N206R, K234R, L229N, Y233H, Y233K, K236R, T230C, Y122H, F74Y, L229A, F174R, Y122A, F219Y, L229G, L229R, T230A, T230S, 1231A, 1231R, I231K, Y233A, Y233R, P238R, Y235F, Y235W, F74Q, P228N, P228A, N217R, S68N, and/or N232R in the primase domain of the wild-type Pyrococcus sp. 12-1 DNA primase with SEQ ID NO: 2.

In one embodiment, the functionally active mutated primase domain comprises at least two amino acid substitutions at positions positionally equivalent to N217 and K234; N217 and K236; N217 and N206; Y122 and Y233; Y122 and N217; Y122 and K234; Y122 and K236; Y122 and N206; Y122 and T230; F74 and N217; F74 and K234; K234 and T230; K236 and T230; N206 and Y233; N206 and T230; or T230 and N217; in the primase domain of the wild-type Pyrococcus sp. 12-1 DNA primase with SEQ ID NO: 2.

In one embodiment, the functionally active mutated primase domain comprises at least three amino acid substitutions at positions positionally equivalent to N217, N206 and Y233; N217, N206 and Y122; or N217, N206 and K234; in the primase domain of the wild-type Pyrococcus sp. 12-1 DNA primase with SEQ ID NO: 2.

In one embodiment, the archaeal DNA primase is from an archaeon of the Pyrococcus genus or from the Thermococcus genus.

In one embodiment, the archaeal DNA primase is from Pyrococcus sp. 12-1, Thermococcus nautili sp. 30-1, Thermococcus sp. CIR10, Thermococcus peptonophilus, or Thermococcus celericrescens.

In one embodiment, the wild-type primase domain of the archaeal DNA primase from Pyrococcus sp. 12-1, Thermococcus nautili sp. 30-1, Thermococcus sp. CIR10, Thermococcus peptonophilus, or Thermococcus celericrescens has an amino acid sequence with any one of SEQ ID NOs: 2-3, 5-16, 18-19, 21 or 23.

In one embodiment, the archaeal DNA primase is from Pyrococcus sp. 12-1.

In one embodiment, the wild-type primase domain of the archaeal DNA primase from Pyrococcus sp. 12-1 has an amino acid sequence with any one of SEQ ID NOs: 2 or 3.

In one embodiment, the primase domain is fused in N terminal or C terminal to a processivity factor, optionally through a linker.

In one embodiment, the processivity factor is a single stranded DNA-binding protein.

In one embodiment, the processivity factor is a single stranded DNA-binding protein from Thermotoga neapolitana.

The present invention also relates to a nucleic acid encoding a functionally active mutated primase domain according to the invention.

The present invention also relates to an expression vector comprising a nucleic acid according to the invention.

The present invention also relates to a host cell comprising an expression vector according to the invention.

The present invention also relates to a method for template-independent synthesis of nucleic acids, comprising iteratively contacting an initiator sequence comprising a 3′-end nucleotide with a free 3′-hydroxyl group, with at least one nucleoside triphosphate, or a combination of nucleoside triphosphates, in the presence of a functionally active mutated primase domain according to the invention, thereby covalently binding said nucleoside triphosphate to the free 3′ hydroxyl group of the 3′ end nucleotide.

In one embodiment, the template-independent synthesis of nucleic acids is carried out at a temperature ranging from about 60° C. to about 95° C.

In one embodiment, the initiator sequence is a single stranded nucleic acid primer, optionally immobilized onto a support.

In one embodiment, the method is:
for template-independent synthesis of nucleic acids with random nucleotide sequence, and the at least one nucleoside triphosphate, or the combination of nucleoside triphosphates, does not comprise terminating nucleoside triphosphates; or
for template-independent sequence-controlled synthesis of nucleic acids, and the at least one nucleoside triphosphate is a terminating nucleoside triphosphate comprising a reversible 3′-blocking group.

The present invention also relates to a kit comprising:
an initiator sequence comprising a 3′-end nucleotide with a free 3′-hydroxyl group, optionally wherein said initiator sequence is immobilized onto a support;
nucleoside triphosphates; and
a functionally active mutated primase domain according to the invention.

### Definitions

“Ab-initio single-stranded nucleic acid synthesis activity” or “template-independent primase activity” means the synthesis of single stranded nucleic acid molecules in absence of both complementary nucleic acid template and initiator sequence, i.e., starting from a single nucleotide.

“DNA primase” refer to enzymes involved in the replication of DNA, belonging to the class of RNA polymerases. They catalyze de novo synthesis of short RNA molecules called primers, typically from 4 to 15 nucleotides in length, from ribonucleoside triphosphates in the presence of a single stranded DNA template. DNA primase activity is required at the replication fork to initiate DNA synthesis by DNA polymerases (Frick & Richardson, 2001. Annu Rev Biochem. 70:39-80).

“Expression vector” refers to a recombinant DNA molecule containing the desired coding nucleic acid sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

“Functionally active fragment”, with reference to an archaeal DNA primase, refers to a fragment or a domain of said archaeal DNA primase which is capable of template-independent terminal nucleotidyl transferase activity. Means and methods to assess the activity of a fragment or a domain of a mutated archaeal DNA primases are well known to the skilled artisan. These include the assays described by Guilliam & Doherty (2017. Methods Enzymol. 591:327-353).

“Positionally equivalent” refers to an amino acid position which is homologous in a given protein by comparison to another protein. For instance, the sequence of the primase domain of the wild-type Pyrococcus sp. 12-1 DNA primase is used herein as reference—unless explicitly stated otherwise—for defining the position(s) of amino acid substitution according to the invention. Hence, in this context, positionally equivalent refers to an amino acid position which is homologous in a given primase domain by comparison to the primase domain of the wild-type Pyrococcus sp. 12-1 DNA primase used as reference. The skilled artisan can readily identify positionally equivalent amino acid residues in the amino acid sequence of a given protein, e.g., on the basis of sequence alignment with the sequence of a protein of reference, and/or by molecular modelling.

“Template-independent terminal nucleotidyl transferase activity” means the addition of nucleoside triphosphates to the 3′ terminus of a nucleic acid molecule, in absence of complementary nucleic acid template. Template-independent nucleic acid synthesis activity can be assessed by means known to the skilled artisan, as described by Guilliam & Doherty (2017. Methods Enzymol. 591:327-353). By way of example, a template-independent nucleic acid synthesis assay can comprise contacting a test enzyme with a single stranded nucleic acid primer as initiator sequence, and dNTPs as building blocks. The single stranded nucleic acid primer can comprise a fluorophore, such as a Cy5 fluorophore in 5′, to allow for visualization. The assay can be carried out at a temperature ranging from 30° C. to 90° C., depending on the thermostability of the test enzyme.

### DETAILED DESCRIPTION

In a first aspect, the present invention relates to a mutated, functionally active primase domain of an archaeal DNA primase; nucleic acids encoding the same; expression vectors comprising the latter; host cells comprising the expression vector; and methods of production of said mutated, functionally active primase domain.

According to the invention, the primase domain is from an archaeal DNA primase belonging to the primase-polymerase (prim-pol) family of the archaeo-eukaryotic primase (AEP) superfamily.

Typically, the primase domain of archaeal DNA primases belonging to the primase-polymerase (prim-pol) family corresponds to the N-terminal domain of the full-length archaeal DNA primase. This N-terminal domain has also been termed “PriS-like domain” in the literature, by opposition to the “PriL-like domain” which corresponds to the C-terminal domain of the archaeal DNA primase.

In one embodiment, the functionally active primase domain comprises or consists of the first N-terminal third of the archaeal DNA primase, such as about the first N-terminal 30 to 35% of the archaeal DNA primase.

In one embodiment, the functionally active primase domain comprises or consists of the first 250 to 355 N-terminal amino acid residues of the archaeal DNA primase, preferably the first 260 to 345, 270 to 335, 280 to 325 or 290 to 315 N-terminal amino acid residues of the archaeal DNA primase.

In one embodiment, the functionally active primase domain may comprise one or several internal loop deletions, that do not affect template-independent terminal nucleotidyl transferase activity. In particular, these loop deletions may be located between amino acid residues positionally equivalents to 90-96, 205-211 and/or 248-254 in the primase domain of Thermococcus nautili sp. 30-1 DNA primase with SEQ ID NO: 5.

The skilled artisan can readily determine which stretches of N-terminal amino acid residues correspond to the primase domain of a given archaeal DNA primase, e.g. by sequence alignment with any one of the primase domains disclosed herein.

According to the invention, the functionally active primase domain is mutated, i.e., it comprises at least one amino acid substitution. The sequence of the primase domain of the wild-type Pyrococcus sp. 12-1 DNA primase is used herein as reference for defining the position(s) of the at least one amino acid substitution, unless explicitly stated otherwise.

The amino acid sequence of the wild-type Pyrococcus sp. 12-1 DNA primase comprises or consists of SEQ ID NO: 1, which represents the amino acid sequence of the protein “p12-17p” from Pyrococcus sp. 12-1 with NCBI Reference Sequence WP_013087941 version 1 of 2019-05-01.

SEQ ID NO: 1
Full-length Pyrococcus sp. 12-1 DNA primase
MRPSDIIIDVYKAIQDHPGAGKLAIELRFYPRPTSEWIIVADIEDKAEEL
HKVLFKNNVLGKKEAYISMALHDFEEVGKKLEKLRELEEERAQKEGRKPR
EVTLRNVQGEATGKVHKTVSKYTLTLVVDIDVEEIHKSKVVESEEKAFEL
AKRAWDELKPKLEGIGVKPRYVFFTGGGVQLWFVAPGLEPIEVIDRASRV
IPPVLNAMLPEGYSVDNIFDRARIVRVPLTINYKYKTPDERPLEIRGRLI
EFNDVRTPLGEVLDKLEAYAKEHGISLVTPSQARFIGTVGRYEVDKGDFR
VLAERLVQELAPWFRRVKEKGGSRHHLVNAIAAYIARNTNLTFEDLAKIW
EIVHAELVRLGLEDPNDWSNRYHTIKDVYEKLQAGTFLGTRAYMLKYLNM
MSEEEIVEILRAVKRALFPYLKPVNAGFVSKFVDEPYGRDEAPKKWEDVD
EDRRKAVGVFFIRALAFETAEYIYVEDLPKPKTFSIKSHSEDGKEKLRFN
EALWQSFLSWLGVQEGEPFDRVDFENLLIEKFGITEHELRSIYFSKVLYL
LTPEGMRMPKCIVEFLKELADEGNLSDEKVAHLAHWIKYFAKPLRHSTTS
IMLRAQGKPVDMRMAVWAKIVEFFAEDDDVAKRLVSVFKKAYEEAEPPFP
CFGVKECPFFPGHKGCPFIAPKRNEILAVSLVDVQIHGTDGIVVVVGGGE
GTKEFVLRGRVEWVKVSKKGVRYPIAEWFLDVFAKEFLALPEPPAWDPGD
VTEILKSRARVVKSQLDEFDEYYEHFIDWLRSENSRGILPYENADSSHLF
IKGKMIGIPPRLAEDFYKIDLGISGKKFKEMLIRKLGKHFLGKKPVWIRL
TSGEKNGVNCYFVSLEWFRDVVGEPNIKEVEAGEELGEFGFSYEEGEA

The amino acid sequence of the wild-type primase domain of the Pyrococcus sp. 12-1 DNA primase (herein termed “PolpP12_{Δ297-898}”) is as set forth in SEQ ID NO: 2.

SEQ ID NO: 2-PolpP12_{Δ297-898}

MRPSDIIIDVYKAIQDHPGAGKLAIELRFYPRPTSEWIIVADIEDKAEE
LHKVLFKNNVLGKKEAYISMALHDFEEVGKKLEKLRELEEERAQKEGRK
PREVTLRNVQGEATGKVHKTVSKYTLTLVVDIDVEEIHKSKVVESEEKA
FELAKRAWDELKPKLEGIGVKPRYVFFTGGGVQLWFVAPGLEPIEVIDR
ASRVIPPVLNAMLPEGYSVDNIFDRARIVRVPLTINYKYKTPDERPLEI
RGRLIEFNDVRTPLGEVLDKLEAYAKEHGISLVTPSQARFIGTVGRYEV
DK

Alternatively, the amino acid sequence of the wild-type primase domain of the Pyrococcus sp. 12-1 DNA primase (herein termed “PolpP12_{Δ7-920297-898}”, with a loop deletion as defined above) is as set forth in SEQ ID NO: 3.

SEQ ID NO: 3-PolpP12_{Δ87-92Δ297-898}

MRPSDIIIDVYKAIQDHPGAGKLAIELRFYPRPTSEWIIVADIEDKAEE
LHKVLFKNNVLGKKEAYISMALHDFEEVGKKLEKLREQKEGRKPREVTL
RNVQGEATGKVHKTVSKYTLTLVVDIDVEEIHKSKVVESEEKAFELAKR
AWDELKPKLEGIGVKPRYVFFTGGGVQLWFVAPGLEPIEVIDRASRVIP
PVLNAMLPEGYSVDNIFDRARIVRVPLTINYKYKTPDERPLEIRGRLIE
FNDVRTPLGEVLDKLEAYAKEHGISLVTPSQARFIGTVGRYEVDK

In one embodiment, the functionally active primase domain is the primase domain of an archaeal DNA primase from an archaeon of the Pyrococcus genus. The Pyrococcus genus comprises several species among which, without limitations, Pyrococcus abyssi, Pyrococcus endeavori, Pyrococcus furiosus, Pyrococcus glycovorans, Pyrococcus horikoshii, Pyrococcus kukulkanii, Pyrococcus woesei, and Pyrococcus yayanosii. The Pyrococcus genus also comprises several unclassified strains, among which, without limitation, Pyrococcus sp. 12-1, Pyrococcus sp. 121, Pyrococcus sp. 303, Pyrococcus sp. 304, Pyrococcus sp. 312, Pyrococcus sp. 32-4, Pyrococcus sp. 321, Pyrococcus sp. 322, Pyrococcus sp. 323, Pyrococcus sp. 324, Pyrococcus sp. 95-12-1, Pyrococcus sp. AV5, Pyrococcus sp. Ax99-7, Pyrococcus sp. C2, Pyrococcus sp. EX2, Pyrococcus sp. Fla95-Pc, Pyrococcus sp. GB-3A, Pyrococcus sp. GB-D, Pyrococcus sp. GBD, Pyrococcus sp. GI-H, Pyrococcus sp. GI-J, Pyrococcus sp. GIL, Pyrococcus sp. HT3, Pyrococcus sp. JT1, Pyrococcus sp. LMO-A29, Pyrococcus sp. LMO-A30, Pyrococcus sp. LMO-A31, Pyrococcus sp. LMO-A32, Pyrococcus sp. LMO-A33, Pyrococcus sp. LMO-A34, Pyrococcus sp. LMO-A35, Pyrococcus sp. LMO-A36, Pyrococcus sp. LMO-A37, Pyrococcus sp. LMO-A38, Pyrococcus sp. LMO-A39, Pyrococcus sp. LMO-A40, Pyrococcus sp. LMO-A41, Pyrococcus sp. LMO-A42, Pyrococcus sp. M24D13, Pyrococcus sp. MA2.31, Pyrococcus sp. MA2.32, Pyrococcus sp. MA2.34, Pyrococcus sp. MV1019, Pyrococcus sp. MV4, Pyrococcus sp. MV7, Pyrococcus sp. MZ14, Pyrococcus sp. MZ4, Pyrococcus sp. NA2, Pyrococcus sp. NS102-T, Pyrococcus sp. P12.1, Pyrococcus sp. PK 5017, Pyrococcus sp. STO4, Pyrococcus sp. ST700, Pyrococcus sp. Tc-2-70, Pyrococcus sp. Tc95-7C-I, Pyrococcus sp. TC95-7C-S, Pyrococcus sp. Tc95_6, Pyrococcus sp. V211, Pyrococcus sp. V212, Pyrococcus sp. V221, Pyrococcus sp. V222, Pyrococcus sp. V231, Pyrococcus sp. V232, Pyrococcus sp. V61, Pyrococcus sp. V62, Pyrococcus sp. V63, Pyrococcus sp. V72, Pyrococcus sp. V73, Pyrococcus sp. VB112, Pyrococcus sp. VB113, Pyrococcus sp. VB81, Pyrococcus sp. VB82, Pyrococcus sp. VB83, Pyrococcus sp. VB85, Pyrococcus sp. VB86, and Pyrococcus sp. VB93.

In one embodiment, the functionally active primase domain is the primase domain of an archaeal DNA primase from an archaeon of the Thermococcus genus. The Thermococcus genus comprises several species among which, without limitations, Thermococcus acidaminovorans, Thermococcus aegaeus, Thermococcus aggregans, Thermococcus alcaliphilus, Thermococcus atlanticus, Thermococcus barophilus, Thermococcus barossii, Thermococcus celer, Thermococcus celericrescens, Thermococcus chitonophagus, Thermococcus cleftensis, Thermococcus coalescens, Thermococcus eurythermalis, Thermococcus fumicolans, Thermococcus gammatolerans, Thermococcus gorgonarius, Thermococcus guaymasensis, Thermococcus hydrothermalis, Thermococcus indicus, Thermococcus kodakarensis, Thermococcus litoralis, Thermococcus marinus, Thermococcus mexicalis, Thermococcus nautili, Thermococcus onnurineus, Thermococcus pacificus, Thermococcus paralvinellae, Thermococcus peptonophilus, Thermococcus piezophilus, Thermococcus prieurii, Thermococcus profundus, Thermococcus radiotolerans, Thermococcus sibiricus, Thermococcus siculi, Thermococcus stetteri, Thermococcus thioreducens, Thermococcus waimanguensis, Thermococcus waiotapuensis, and Thermococcus zilligii. The Thermococcus genus also comprises several unclassified strains among which, without limitation, Thermococcus sp. AEPII 1a, Thermococcus sp. 101 C5, Thermococcus sp. 11N.A5, Thermococcus sp. 12-4, Thermococcus sp. 13-2, Thermococcus sp. 13-3, Thermococcus sp. 1519, Thermococcus sp. 175, Thermococcus sp. 17S1, Thermococcus sp. 17S2, Thermococcus sp. 17S3, Thermococcus sp. 17S4, Thermococcus sp. 17S5, Thermococcus sp. 17S6, Thermococcus sp. 17S8, Thermococcus sp. 18S1, Thermococcus sp. 18S2, Thermococcus sp. 18S3, Thermococcus sp. 18S4, Thermococcus sp. 18S5, Thermococcus sp. 21-1, Thermococcus sp. 21S1, Thermococcus sp. 21S2, Thermococcus sp. 21S3, Thermococcus sp. 21S4, Thermococcus sp. 21S5, Thermococcus sp. 21S6, Thermococcus sp. 21S7, Thermococcus sp. 21S8, Thermococcus sp. 21S9, Thermococcus sp. 23-1, Thermococcus sp. 23-2, Thermococcus sp. 2319x1, Thermococcus sp. 26-2, Thermococcus sp. 26-3, Thermococcus sp. 26/2, Thermococcus sp. 28-1, Thermococcus sp. 29-1, Thermococcus sp. 300-Tc, Thermococcus sp. 31-1, Thermococcus sp. 31-3, Thermococcus sp. 40_45. Thermococcus sp. 4557, Thermococcus sp. 5-1, Thermococcus sp. 5-4, Thermococcus sp. 70-4-2, Thermococcus sp. 7324, Thermococcus sp. 83-5-2, Thermococcus sp. 9N2, Thermococcus sp. 9N2.20, Thermococcus sp. 9N2.21, Thermococcus sp. 9N3, Thermococcus sp. 9oN-7, Thermococcus sp. A4, Thermococcus sp. AF1T14.13, Thermococcus sp. AF1T1423, Thermococcus sp. AF1T20.11, Thermococcus sp. AF1T6.10, Thermococcus sp. AF1T6.12, Thermococcus sp. AF1T6.63, Thermococcus sp. AF2T511, Thermococcus sp. Ag85-vw, Thermococcus sp. AM4, Thermococcus sp. AMT11, Thermococcus sp. AMT7, Thermococcus sp. Anhete70-I78, Thermococcus sp. Anhete70-SCI, Thermococcus sp. Anhete85-I78, Thermococcus sp. Anhete85-SCI, Thermococcus sp. AT1273, Thermococcus sp. AV1, Thermococcus sp. AV2, Thermococcus sp. AV3, Thermococcus sp. AV6, Thermococcus sp. AV7, Thermococcus sp. AV9, Thermococcus sp. AV10, Thermococcus sp. AV11, Thermococcus sp. AV13, Thermococcus sp. AV14, Thermococcus sp. AV15, Thermococcus sp. AV16, Thermococcus sp. AV17, Thermococcus sp. AV18, Thermococcus sp. AV20, Thermococcus sp. AV21, Thermococcus sp. AV22, Thermococcus sp. Ax00-17, Thermococcus sp. Ax00-27, Thermococcus sp. Ax00-39, Thermococcus sp. Ax00-45, Thermococcus sp. Ax01-2, Thermococcus sp. Ax01-3, Thermococcus sp. Ax01-37, Thermococcus sp. Ax01-39, Thermococcus sp. Ax01-61, Thermococcus sp. Ax01-62, Thermococcus sp. Ax01-65, Thermococcus sp. Ax98-43, Thermococcus sp. Ax98-46, Thermococcus sp. Ax98-48, Thermococcus sp. Ax99-47, Thermococcus sp. Ax99-57, Thermococcus sp. Ax99-67, Thermococcus sp. AXTV6, Thermococcus sp. B1, Thermococcus sp. B1001, Thermococcus sp. B4, Thermococcus sp. BHI60a21, Thermococcus sp. BHI80a28, Thermococcus sp. BHI80a40, Thermococcus sp. Bubb.Bath, Thermococcus sp. BX13, Thermococcus sp. CAR-80, Thermococcus sp. Champagne, Thermococcus sp. CIR10, Thermococcus sp. CKU-1, Thermococcus sp. CKU-199, Thermococcus sp. CL2, Thermococcus sp. CMI, Thermococcus sp. CNR-5, Thermococcus sp. CX1, Thermococcus sp. CX2, Thermococcus sp. CX3, Thermococcus sp. CX4, Thermococcus sp. CYA, Thermococcus sp. Dex80a71, Thermococcus sp. Dex80a75, Thermococcus sp. DS-1, Thermococcus sp. DS1, Thermococcus sp. DT4, Thermococcus sp. ENR5, Thermococcus sp. EP1, Thermococcus sp. ES5, Thermococcus sp. ES6, Thermococcus sp. ES7, Thermococcus sp. ES8, Thermococcus sp. ES9, Thermococcus sp. ES10, Thermococcus sp. ES11, Thermococcus sp. ES12, Thermococcus sp. ES13, Thermococcus sp. EXT12c, Thermococcus sp. EXT9, Thermococcus sp. Fe85_1_2, Thermococcus sp. GB18, Thermococcus sp. GB20, Thermococcus sp. GE8, Thermococcus sp. Gorda2, Thermococcus sp. Gorda3, Thermococcus sp. Gorda4, Thermococcus sp. Gorda5, Thermococcus sp. Gorda6, Thermococcus sp. GR2, Thermococcus sp. GR4, Thermococcus sp. GR5, Thermococcus sp. GR6, Thermococcus sp. GR7, Thermococcus sp. GT, Thermococcus sp. GU5L5, Thermococcus sp. HJ21, Thermococcus sp. IRI33, Thermococcus sp. IRI35c, Thermococcus sp. IRI48, Thermococcus sp. JCM 11816, Thermococcus sp. JDF-3, Thermococcus sp. JdF3, Thermococcus sp. JdFR-02, Thermococcus sp. KBA1, Thermococcus sp. KI, Thermococcus sp. KS-8, Thermococcus sp. LMO-A1, Thermococcus sp. LMO-A2, Thermococcus sp. LMO-A3, Thermococcus sp. LMO-A4, Thermococcus sp. LMO-A5, Thermococcus sp. LMO-A6, Thermococcus sp. LMO-A7, Thermococcus sp. LMO-A8, Thermococcus sp. LMO-A9, Thermococcus sp. LS1, Thermococcus sp. LS2, Thermococcus sp. M36, Thermococcus sp. M39, Thermococcus sp. MA2.27, Thermococcus sp. MA2.28, Thermococcus sp. MA2.29, Thermococcus sp. MA2.33, Thermococcus sp. MAR1, Thermococcus sp. MAR2, Thermococcus sp. MCR132, Thermococcus sp. MCR133, Thermococcus sp. MCR134, Thermococcus sp. MCR135, Thermococcus sp. MCR175, Thermococcus sp. MV1, Thermococcus sp. MV2, Thermococcus sp. MV3, Thermococcus sp. MV5, Thermococcus sp. MV10, Thermococcus sp. MV11, Thermococcus sp. MV12, Thermococcus sp. MV13, Thermococcus sp. MV1031, Thermococcus sp. MV1049, Thermococcus sp. MV1083, Thermococcus sp. MV1092, Thermococcus sp. MV1099, Thermococcus sp. MZ1, Thermococcus sp. MZ2, Thermococcus sp. MZ3, Thermococcus sp. MZ5, Thermococcus sp. MZ6, Thermococcus sp. MZ7, Thermococcus sp. MZ8, Thermococcus sp. MZ9, Thermococcus sp. MZ10, Thermococcus sp. MZ11, Thermococcus sp. MZ12, Thermococcus sp. MZ13, Thermococcus sp. NS85-T, Thermococcus sp. P6, Thermococcus sp. Pd70, Thermococcus sp. Pd85, Thermococcus sp. PK, Thermococcus sp. PK(2011), Thermococcus sp. Rt3, Thermococcus sp. SB611, Thermococcus sp. SN531, Thermococcus sp. SRB55_1, Thermococcus sp. SRB70_1, Thermococcus sp. SRB70_10, Thermococcus sp. SY113, Thermococcus sp. Tc-1-70, Thermococcus sp. Tc-1-85, Thermococcus sp. Tc-1-95, Thermococcus sp. Tc-2-85, Thermococcus sp. Tc-2-95, Thermococcus sp. Tc-365-70, Thermococcus sp. Tc-365-85, Thermococcus sp. Tc-365-95, Thermococcus sp. Tc-4-70, Thermococcus sp. Tc-4-85, Thermococcus sp. Tc-I-70, Thermococcus sp. Tc-I-85, Thermococcus sp. Tc-S-70, Thermococcus sp. Tc-S-85, Thermococcus sp. Tc55_1, Thermococcus sp. Tc55_12, Thermococcus sp. Tc70-4C-I, Thermococcus sp. Tc70-4C-S, Thermococcus sp. Tc70-7C-I, Thermococcus sp. Tc70-7C-S, Thermococcus sp. Tc70-CRC-I, Thermococcus sp. Tc70-CRC-S, Thermococcus sp. Tc70-MC-S, Thermococcus sp. Tc70-SC-I, Thermococcus sp. Tc70-SC-S, Thermococcus sp. Tc70-vw, Thermococcus sp. Tc70_1, Thermococcus sp. Tc70_10, Thermococcus sp. Tc70_11, Thermococcus sp. Tc70_12, Thermococcus sp. Tc70_20, Thermococcus sp. Tc70_6, Thermococcus sp. Tc70_9, Thermococcus sp. Tc85-0 age SC, Thermococcus sp. Tc85-4C-I, Thermococcus sp. Tc85-4C-S, Thermococcus sp. Tc85-7C-S, Thermococcus sp. Tc85-CRC-I, Thermococcus sp. Tc85-CRC-S, Thermococcus sp. Tc85-MC-I, Thermococcus sp. Tc85-MC-S, Thermococcus sp. Tc85-SC-I, Thermococcus sp. Tc85-SC-ISCS, Thermococcus sp. Tc85-SC-S, Thermococcus sp. Tc85_1, Thermococcus sp. Tc85_10, Thermococcus sp. Tc85_11, Thermococcus sp. Tc85_12, Thermococcus sp. Tc85_13, Thermococcus sp. Tc85_19, Thermococcus sp. Tc85_2, Thermococcus sp. Tc85_20, Thermococcus sp. Tc85_9, Thermococcus sp. Tc95-CRC-I, Thermococcus sp. Tc95-CRC-S, Thermococcus sp. Tc95-MC-I, Thermococcus sp. Tc95-MC-S, Thermococcus sp. Tc95-SC-S, Thermococcus sp. TK1, Thermococcus sp. TKM 55-W7-A, Thermococcus sp. TM1, Thermococcus sp. TP-33, Thermococcus sp. TP-37, Thermococcus sp. TS3, Thermococcus sp. TVG2, and Thermococcus sp. vp197.

In one embodiment, the functionally active primase domain is the primase domain of the archaeal DNA primase from Pyrococcus sp. 12-1, Thermococcus nautili sp. 30-1, Thermococcus sp. CIR10, Thermococcus peptonophilus, or Thermococcus celericrescens.

In one embodiment, the functionally active primase domain is the primase domain of the archaeal DNA primase from Pyrococcus sp. 12-1.

In one embodiment, the functionally active primase domain shares at least 80%, 85%, 90%, 95%, 96%, 97%, 98%, 99%, 99.5%, 99.9% or 100% of sequence identity with the primase domain of from Pyrococcus sp. 12-1 with SEQ ID NO: 2 or SEQ ID NO: 3.

In one embodiment, the functionally active primase domain shares at least 80%, 99%, 99.1%, 99.2%, 99.3%, 99.4%, 99.5%, 99.6%, 99.7%, 99.8%, 99.9% or 100% of sequence identity with the primase domain of from Pyrococcus sp. 12-1 with SEQ ID NO: 2 or SEQ ID NO: 3.

In one embodiment, the functionally active primase domain is the primase domain of the archaeal DNA primase from Pyrococcus sp. 12-1, with SEQ ID NO: 2 or SEQ ID NO: 3.

In another embodiment, the functionally active primase domain is the primase domain of the archaeal DNA primase from Thermococcus nautili sp. 30-1.

The amino acid sequence of the wild-type Thermococcus nautili sp. 30-1 DNA primase comprises or consists of SEQ ID NO: 4, which represents the amino acid sequence of the protein “tn2-12p” from Thermococcus nautili sp. 30-1 with NCBI Reference Sequence WP_013087990 version 1 of 2019-05-01.

SEQ ID NO: 4-Full-length Thermococcus nautili sp. 30-1 DNA primase

MSSLRPSSIIIDIYKVIQDHPDSSRWAIELRFLPKPISSEWIFITDIEERASEIDKVLT
KYNIMKKKDAYVSMAIHDFEKVTAKLKRVQEEENKKATEGERRLRRITLDKIQ
GDSETTSGFTLALVVDIDNTKIHDTRIIENEEEAFEASKREWEALKPKLQELGFL
PRWILYTGGGLQLWFVSDKLEPISVIDRASEIIPNIMNGVNGVKGLLSEGFKADN
IFDPARIVRAPLTFNHKYRTIIKDEDGTERVVPTQVKGRVIEFNDVRISLTEFLDR
LEAYAKEKGIPLEKPTKRKFLELASKRYEVTSSNFEALAERLFTELRPWWEIAK
EKGWSRHHLTMGIATYILRNTNLTPEQLIGSENSPGLWELVFAKLVEAGLEDPD
DWKNRASTIRDAYKKIESGKKVATKAYLRKYIEGLSEEDAVQILLSVKRALLPY
LKAVDVKRISKYSARPYEITEEIPKSWEDIDENRKKATGVWYIDFLALETANDIY
FEDLPKPPVFYIRFVEKNKEKFKLNETLYHSFLTWLGITEGEPLDRTELIDLLVEK
FGFTVEDLKAIYYRKILTLLKPEGMRTPKCIQEFLFELATEGNLPEDKIRHLAHW
VKFYARPLRHSTTSVLLKGRGKPVDMRLAIWAKVVEFFAEDDEVAEELITTFK
KAYMGAEPPFPCIGAESCPFYPDHRACPFIVPKRKEVLPVSIVDVQLHGSDGIVV
LVGGPTEVTAFTLEGKVEWIKTTKKTVKYPIAEWFLDRFAKEYLSLPEAPSWW
KLEEVTEILKSRARVVKSQFDKFEDYLEQFIEWLQKENSRRGILPYEKADENHL
FIKGEWVGIPPGFAREFYSGELLIGGPTFRRMLEQKLGKDYRKMSAKIHLNTGL
KDKRNCYFVSVEWFRKHVGEPNIQEITSEGDVSFDGLSYDDEEEGVVG

The amino acid sequence of the wild-type primase domain of the Thermococcusnautili sp. 30-1 DNA primase (herein termed “PolpTN2_{Δ311-923}”) is as set forth in SEQ ID NO: 5.

SEQ ID NO: 5-PolpTN2_{Δ311-923}

MSSLRPSSIIIDIYKVIQDHPDSSRWAIELRFLPKPISSEWIFITDIEERASEIDKVLT
KYNIMKKKDAYVSMAIHDFEKVTAKLKRVQEEENKKATEGERRLRRITLDKIQ
GDSETTSGFTLALVVDIDNTKIHDTRIIENEEEAFEASKREWEALKPKLQELGFL
PRWILYTGGGLQLWFVSDKLEPISVIDRASEIIPNIMNGVNGVKGLLSEGFKADN
IFDPARIVRAPLTFNHKYRTIIKDEDGTERVVPTQVKGRVIEFNDVRISLTEFLDR
LEAYAKEKGIPLEKPTKRKFLELASKRYEVTS

Alternatively, the amino acid sequence of the wild-type primase domain of the Thermococcus nautili sp. 30-1 DNA primase (herein termed “PolpTN2_{Δ90-96Δ311-923}”), with a loop deletion as defined above, is as set forth in SEQ ID NO: 6.

SEQ ID NO: 6-PolpTN2_{Δ90-96Δ311-923}

MSSLRPSSIIIDIYKVIQDHPDSSRWAIELRFLPKPISSEWIFITDIEERASEIDKVLT
KYNIMKKKDAYVSMAIHDFEKVTAKLKRVQETEGERRLRRITLDKIQGDSETTS
GFTLALVVDIDNTKIHDTRIIENEEEAFEASKREWEALKPKLQELGFLPRWILYT
GGGLQLWFVSDKLEPISVIDRASEIIPNIMNGVNGVKGLLSEGFKADNIFDPARI
VRAPLTFNHKYRTIIKDEDGTERVVPTQVKGRVIEFNDVRISLTEFLDRLEAYAK
EKGIPLEKPTKRKFLELASKRYEVTS

Alternatively, the amino acid sequence of the wild-type primase domain of the Thermococcus nautili sp. 30-1 DNA primase (herein termed “PolpTN2_{Δ205-2110311-923}”), with a loop deletion as defined above, is as set forth in SEQ ID NO: 7.

SEQ ID NO: 7-PolpTN2_{Δ205-211Δ311-923}

MSSLRPSSIIIDIYKVIQDHPDSSRWAIELRFLPKPISSEWIFITDIEERASEIDKVLT
KYNIMKKKDAYVSMAIHDFEKVTAKLKRVQEEENKKATEGERRLRRITLDKIQ
GDSETTSGFTLALVVDIDNTKIHDTRIIENEEEAFEASKREWEALKPKLQELGFL
PRWILYTGGGLQLWFVSDKLEPISVIDRASEIIPNIMNGLLSEGFKADNIFDPARI
VRAPLTFNHKYRTIIKDEDGTERVVPTQVKGRVIEFNDVRISLTEFLDRLEAYAK
EKGIPLEKPTKRKFLELASKRYEVTS

Alternatively, the amino acid sequence of the wild-type primase domain of the Thermococcus nautili sp. 30-1 DNA primase (herein termed “PolpTN2_{Δ248-254Δ311-923}”), with a loop deletion as defined above, is as set forth in SEQ ID NO: 8.

SEQ ID NO: 8-PolpTN2_{Δ248-254Δ311-923}

MSSLRPSSIIIDIYKVIQDHPDSSRWAIELRFLPKPISSEWIFITDIEERASEIDKVLT
KYNIMKKKDAYVSMAIHDFEKVTAKLKRVQEEENKKATEGERRLRRITLDKIQ
GDSETTSGFTLALVVDIDNTKIHDTRIIENEEEAFEASKREWEALKPKLQELGFL
PRWILYTGGGLQLWFVSDKLEPISVIDRASEIIPNIMNGVNGVKGLLSEGFKADN
IFDPARIVRAPLTFNHKYRTIIKDEDPTQVKGRVIEFNDVRISLTEFLDRLEAYAK
EKGIPLEKPTKRKFLELASKRYEVTS

Alternatively, the amino acid sequence of the wild-type primase domain of the Thermococcus nautili sp. 30-1 DNA primase (herein termed “PolpTN2_{Δ243-254Δ311-923}”), with a loop deletion as defined above, is as set forth in SEQ ID NO: 9.

SEQ ID NO: 9-PolpTN2_{Δ243-254Δ311-923}

MSSLRPSSIIIDIYKVIQDHPDSSRWAIELRFLPKPISSEWIFITDIEERASEIDKVLT
KYNIMKKKDAYVSMAIHDFEKVTAKLKRVQEEENKKATEGERRLRRITLDKIQ
GDSETTSGFTLALVVDIDNTKIHDTRIIENEEEAFEASKREWEALKPKLQELGFL
PRWILYTGGGLQLWFVSDKLEPISVIDRASEIIPNIMNGVNGVKGLLSEGFKADN
IFDPARIVRAPLTFNHKYRTPTQVKGRVIEFNDVRISLTEFLDRLEAYAKEKGIPL
EKPTKRKFLELASKRYEVTS

Alternatively, the amino acid sequence of the wild-type primase domain of the Thermococcus nautili sp. 30-1 DNA primase (herein termed “PolpTN2_{Δ90-96Δ205-2110311-923}”), with two loop deletions as defined above, is as set forth in SEQ ID NO: 10.

SEQ ID NO: 10-PolpTN2_{Δ90-96Δ205-211Δ311-923}

MSSLRPSSIIIDIYKVIQDHPDSSRWAIELRFLPKPISSEWIFITDIEERASEIDKVLT
KYNIMKKKDAYVSMAIHDFEKVTAKLKRVQETEGERRLRRITLDKIQGDSETTS
GFTLALVVDIDNTKIHDTRIIENEEEAFEASKREWEALKPKLQELGFLPRWILYT
GGGLQLWFVSDKLEPISVIDRASEIIPNIMNGLLSEGFKADNIFDPARIVRAPLTF
NHKYRTIIKDEDGTERVVPTQVKGRVIEFNDVRISLTEFLDRLEAYAKEKGIPLE
KPTKRKFLELASKRYEVTS

Alternatively, the amino acid sequence of the wild-type primase domain of the Thermococcus nautili sp. 30-1 DNA primase (herein termed “PolpTN2_{Δ90-96Δ248-254Δ311-923}”), with two loop deletions as defined above, is as set forth in SEQ ID NO: 11.

SEQ ID NO: 11-PolpTN2_{Δ90-96Δ248-254Δ311-923}

MSSLRPSSIIIDIYKVIQDHPDSSRWAIELRFLPKPISSEWIFITDIEERASEIDKVLT
KYNIMKKKDAYVSMAIHDFEKVTAKLKRVQETEGERRLRRITLDKIQGDSETTS
GFTLALVVDIDNTKIHDTRIIENEEEAFEASKREWEALKPKLQELGFLPRWILYT
GGGLQLWFVSDKLEPISVIDRASEIIPNIMNGVNGVKGLLSEGFKADNIFDPARI
VRAPLTFNHKYRTIIKDEDPTQVKGRVIEFNDVRISLTEFLDRLEAYAKEKGIPLE
KPTKRKFLELASKRYEVTS

Alternatively, the amino acid sequence of the wild-type primase domain of the Thermococcusnautili sp. 30-1 DNA primase (herein termed “PolpTN2 _{Δ90-96Δ243-254Δ311-923}”), with two loop deletions as defined above, is as set forth in SEQ ID NO: 12.

SEQ ID NO: 12-PolpTN2_{Δ90-96Δ243-254Δ311-923}

MSSLRPSSIIIDIYKVIQDHPDSSRWAIELRFLPKPISSEWIFITDIEERASEIDKVLT
KYNIMKKKDAYVSMAIHDFEKVTAKLKRVQETEGERRLRRITLDKIQGDSETTS
GFTLALVVDIDNTKIHDTRIIENEEEAFEASKREWEALKPKLQELGFLPRWILYT
GGGLQLWFVSDKLEPISVIDRASEIIPNIMNGVNGVKGLLSEGFKADNIFDPARI
VRAPLTFNHKYRTPTQVKGRVIEFNDVRISLTEFLDRLEAYAKEKGIPLEKPTKR
KFLELASKRYEVTS

Alternatively, the amino acid sequence of the wild-type primase domain of the Thermococcusnautili sp. 30-1 DNA primase (herein termed “PolpTN2 _{Δ205-211Δ248-254Δ311-923}”), with two loop deletions as defined above, is as set forth in SEQ ID NO: 13.

SEQ ID NO: 13-PolpTN2_{Δ205-211Δ248-254Δ311-923}

MSSLRPSSIIIDIYKVIQDHPDSSRWAIELRFLPKPISSEWIFITDIEERASEIDKVLT
KYNIMKKKDAYVSMAIHDFEKVTAKLKRVQEEENKKATEGERRLRRITLDKIQ
GDSETTSGFTLALVVDIDNTKIHDTRIIENEEEAFEASKREWEALKPKLQELGFL
PRWILYTGGGLQLWFVSDKLEPISVIDRASEIIPNIMNGLLSEGFKADNIFDPARI
VRAPLTFNHKYRTIIKDEDPTQVKGRVIEFNDVRISLTEFLDRLEAYAKEKGIPLE
KPTKRKFLELASKRYEVTS

Alternatively, the amino acid sequence of the wild-type primase domain of the Thermococcus nautili sp. 30-1 DNA primase (herein termed “PolpTN2_{Δ05-211Δ243-254Δ311-923}”), with two loop deletions as defined above, is as set forth in SEQ ID NO: 14.

SEQ ID NO: 14-PolpTN2_{Δ205-211Δ243-254Δ311-923}

MSSLRPSSIIIDIYKVIQDHPDSSRWAIELRFLPKPISSEWIFITDIEERASEIDKVLT
KYNIMKKKDAYVSMAIHDFEKVTAKLKRVQEEENKKATEGERRLRRITLDKIQ
GDSETTSGFTLALVVDIDNTKIHDTRIIENEEEAFEASKREWEALKPKLQELGFL
PRWILYTGGGLQLWFVSDKLEPISVIDRASEIIPNIMNGLLSEGFKADNIFDPARI
VRAPLTFNHKYRTPTQVKGRVIEFNDVRISLTEFLDRLEAYAKEKGIPLEKPTKR
KFLELASKRYEVTS

Alternatively, the amino acid sequence of the wild-type primase domain of the Thermococcus nautili sp. 30-1 DNA primase (herein termed “PolpTN2_{Δ90-96Δ205-211Δ248-254Δ311-923}”), with three loop deletions as defined above, is as set forth in SEQ ID NO: 15.

SEQ ID NO: 15-PolpTN2_{Δ90-96Δ205-211Δ248-254Δ311-923}

MSSLRPSSIIIDIYKVIQDHPDSSRWAIELRFLPKPISSEWIFITDIEERASEIDKVLT
KYNIMKKKDAYVSMAIHDFEKVTAKLKRVQETEGERRLRRITLDKIQGDSETTS
GFTLALVVDIDNTKIHDTRIIENEEEAFEASKREWEALKPKLQELGFLPRWILYT
GGGLQLWFVSDKLEPISVIDRASEIIPNIMNGLLSEGFKADNIFDPARIVRAPLTF
NHKYRTIIKDEDPTQVKGRVIEFNDVRISLTEFLDRLEAYAKEKGIPLEKPTKRK
FLELASKRYEVTS

Alternatively, the amino acid sequence of the wild-type primase domain of the Thermococcus nautili sp. 30-1 DNA primase (herein termed “PolpTN2_{Δ90-96Δ205-211Δ243-254Δ311-923}”), with three loop deletions as defined above, is as set forth in SEQ ID NO: 16.

SEQ ID NO: 16-PolpTN2_{Δ90-96Δ205-211Δ243-254Δ311-923}

MSSLRPSSIIIDIYKVIQDHPDSSRWAIELRFLPKPISSEWIFITDIEERASEIDKVLT
KYNIMKKKDAYVSMAIHDFEKVTAKLKRVQETEGERRLRRITLDKIQGDSETTS
GFTLALVVDIDNTKIHDTRIIENEEEAFEASKREWEALKPKLQELGFLPRWILYT
GGGLQLWFVSDKLEPISVIDRASEIIPNIMNGLLSEGFKADNIFDPARIVRAPLTF
NHKYRTPTQVKGRVIEFNDVRISLTEFLDRLEAYAKEKGIPLEKPTKRKFLELAS
KRYEVTS

In one embodiment, the functionally active primase domain is the primase domain of the archaeal DNA primase from Thermococcus sp. CIR10.

The amino acid sequence of the Thermococcus sp. CIR10 DNA primase comprises or consists of SEQ ID NO: 17, which represents the amino acid sequence of the protein “primase/polymerase” from Thermococcus sp. CIR10 with NCBI Reference Sequence WP_015243587 version 1 of 2016-06-18.

SEQ ID NO: 17-Full length Thermococcus sp. CIR10 DNA primase

MSGREFKRPSDVIIDIYKVIQDHPEAGRLAIEFRFYPYPTSEWILLNDIEDKAREID
KVLFKNNILGKKEAYISMAIHDFDEVTKKLEKLQELEHEKAQKEGRQPKEITLR
HVQGEATGKIHTTVSSYTLTLVVDIDVNEIHDSKAVESEEKALEVSKRAWEVLK
PNLEELGIKPRYVFFTGGGIQLWFVAPEPENISVIDKAAEIIPPVLNTLLPEGYSV
DNIFDRARIVRVPFTVNYKYKTPDGKPLELRGRLLEFNDVRTPLGDILEKLEAY
AKGHKISLGSTSRSGKFRGVAGRYEVKKENFEELAKRLVEELAPWFKKIKERG
GSRHHLVNAIAAYIARNTNLTEEDLLGKDQEDGTHVVGLWELVHSKLVELGLE
DPGDWSNRYHTIKDVYEKLYAGTTLGTRAYMMKYLNVSEEEAIEILRSVKRAL
FPYLHPVNVQVISKFEAKPYSKEEAPTEWEAVDEDRKKAVGIWYIEVLALETA
NYVYIEDLSKPGVFYIVEKVKRTVKVGKKEKGVEVDEYHFNPALWQSFLNWL
GIKEGEPIEREELWNLLLEKFDIKDYELRAIYFRKILHLLSPEGMRRPRCVEEFLR
ELADEGFLSEDKVRHLAHWIKFYAKPLRHSTTSIMLRAKGKPVDMRMAVWAK
VVEFFAEDEETAQGLIETFREAYEQAEPPFPCFGARECPFFQEHRGCPFIAPKRDE
ILAVSLVDVQLHGSDGIVIIVGSEEGTKKFVHKGKVEWQKQGKSKIKYPVAEW
FLDVYAKEFLSLPEAPSWSHEEVTEILKSRARVVRSQLNEFDEYFDNFIDWLRSE
NARGIYPYEKADSSHIFIKGNMIGIPPRLAEDFYRNELGISGRKFKEMLIRELGSY
YLGKKAAWIKLSSGQHNGVNCYFISLDWFKKIVGEPNIKDIEAEGDIGSGGFNY
EEEEGEA

The amino acid sequence of the wild-type primase domain of the Thermococcus sp. CIR10 DNA primase (herein termed “PolpCIR10303-928”) is as set forth in SEQ ID NO: 18.

SEQ ID NO: 18-PolpCIR10_{Δ303-928}

MSGREFKRPSDVIIDIYKVIQDHPEAGRLAIEFRFYPYPTSEWILLNDIEDKAREID
KVLFKNNILGKKEAYISMAIHDFDEVTKKLEKLQELEHEKAQKEGRQPKEITLR
HVQGEATGKIHTTVSSYTLTLVVDIDVNEIHDSKAVESEEKALEVSKRAWEVLK
PNLEELGIKPRYVFFTGGGIQLWFVAPEPENISVIDKAAEIIPPVLNTLLPEGYSV
DNIFDRARIVRVPFTVNYKYKTPDGKPLELRGRLLEFNDVRTPLGDILEKLEAY
AKGHKISLGSTSRSGKFRGVAGRYEVKK

Alternatively, the amino acid sequence of the wild-type primase domain of the Thermococcus sp. CIR10 DNA primase (herein termed “PolpCIR10_{Δ93-98Δ303-928}”), with a loop deletion as defined above, is as set forth in SEQ ID NO: 19.

SEQ ID NO: 19-PolpCIR10_{Δ93-98Δ303-928}

MSGREFKRPSDVIIDIYKVIQDHPEAGRLAIEFRFYPYPTSEWILLNDIEDKAREID
KVLFKNNILGKKEAYISMAIHDFDEVTKKLEKLQEQKEGRQPKEITLRHVQGEA
TGKIHTTVSSYTLTLVVDIDVNEIHDSKAVESEEKALEVSKRAWEVLKPNLEEL
GIKPRYVFFTGGGIQLWFVAPEPENISVIDKAAEIIPPVLNTLLPEGYSVDNIFDR
ARIVRVPFTVNYKYKTPDGKPLELRGRLLEFNDVRTPLGDILEKLEAYAKGHKI
SLGSTSRSGKFRGVAGRYEVKK

In one embodiment, the functionally active primase domain is the primase domain of the archaeal DNA primase from Thermococcus peptonophilus.

The amino acid sequence of the wild-type Thermococcus peptonophilus DNA primase comprises or consists of SEQ ID NO: 20, which represents the amino acid sequence of an “hypothetical protein” from Thermococcus peptonophilus with NCBI Reference Sequence WP_062389070 version 1 of 2016-03-28.

SEQ ID NO: 20-Full-length Thermococcus peptonophilus DNA primase

MSELTPGKVLADVYKVIQDHPEAGRLAIELRFYPVIKSEWVLLNDIEDKARDID
KVLAKQNLIKGKEAYVSMAIHGFEAVKKKLEKLRESVEEGKVRKLGLENVQG
EAKGKVHPTVSNYTLTLVVDVDIEAVHKLKVVEDVDKVFEKAKEGWLALKPV
FEELGVLPRYVFFTGGGLQLWFVAPKLEDIAVIDRASGIVPNVLNALLPEGFVV
DNIFDRARIVRAPLTVNHKYKAPNGARVGVKGRLIEFNDVRVSLSEVLDKLEV
YAKERGIQLGGQEKVRGGRGFVNVRYVVKKEELETLALNLADELIPWFKKVK
ERGGSWHHLVNAIGAYVVRNTNLSLEDLIGKDNPDGTHVVGLWEIVFQRLVEK
SAEDPGDWVNRRNTIKDVYEKHIAGKPLGTRAYLKKYLPVSDEEVVEILMAVR
RALLPFLKEVKKVDSSGFGVAPYKKTAPRSWDEVDEDRKRATGRWYVWKLA
FNTAEYLFTDELPKAGTFYIDVWMKEGKREWMKRFFNEALFRSFIEDGLGYKY
GAPVEREELFERLVEVFNITDEEVRGIYIDAALSLLSPVGMRTPPCIEEFIMEFAA
NGSLSEDKVRHLARWIKLYAKPLKHSTTTTKLVGAGYKVDMRMAVWAKLVE
FFAEDDEVARELVRVFKEEYGAAEPPFTCIGTKTCQFYLNEKMCPFIIPKEKEIL
AVSLIDVQRHESDGLVVIVGGDKEVRTFVKKGNVEWVKKTERREKYPVAEWFI
DVFATEYLSVSPDDLDVDLEEVTDILKSRARVVKSRLNELEDMYEKFVEWLKR
ENAVRGVLPYEKADFNHLFIKGNMIGIPPALAEEFYRFELNIKGSEFREMLEKKL
GFHYTKKAVKLSVGEKKDVRRCYLVSLEWFRKVVGEPNVKDVVMAGDIALS
GLVYYESGEEVVE

The amino acid sequence of the wild-type primase domain of the Thermococcus peptonophilus DNA primase (herein termed “PolpTpep_{Δ295-914}”) is as set forth in SEQ ID NO: 21.

SEQ ID NO: 21-PolpTpep_{Δ295-914}

MSELTPGKVLADVYKVIQDHPEAGRLAIELRFYPVIKSEWVLLNDIEDK
ARDIDKVLAKQNLIKGKEAYVSMAIHGFEAVKKKLEKLRESVEEGKVRK
LGLENVQGEAKGKVHPTVSNYTLTLVVDVDIEAVHKLKVVEDVDKVFEK
AKEGWLALKPVFEELGVLPRYVFFTGGGLQLWFVAPKLEDIAVIDRASG
IVPNVLNALLPEGFVVDNIFDRARIVRAPLTVNHKYKAPNGARVGVKGR
LIEFNDVRVSLSEVLDKLEVYAKERGIQLGGQEKVRGGRGFVNVRYVVK
K

In one embodiment, the functionally active primase domain is the primase domain of the archaeal DNA primase from Thermococcus celericrescens.

The amino acid sequence of the wild-type Thermococcus celericrescens DNA primase comprises or consists of SEQ ID NO: 22, which represents the amino acid sequence of an “hypothetical protein” from Thermococcus celericrescens with NCBI Reference Sequence WP_058937716 version 1 of 2016-01-06.

SEQ ID NO: 22-Full-length Thermococcus celericrescens DNA primase

MSELTPGKVLADVYKVIQDHPEAGRLAIELRFYPVIKSEWVLLNDIEDKARDID
KVLAKKNIINGKEAYVSMAIHDFGAVKKKLEKLREKAEGERARRIGLENVQGE
AKGKVHPTVSNYTLALVVDIDIEEVHKSRVVEDVEAVFERAKKGWLALRPVFE
ELGVLPRYVFFTGGGLQIWFVAPELEDIAVIDRASGIVPGVLNALLPEGFVVDNI
FDRARIVRAPLTVNHKYKAPNGAGLGVKGRLIEFNDVRVSLSEVLDKLEAYAK
EKGIQLGGQERASGVRVFGKVRYEVKKERLETLALNLADELAPWFKKVKERG
GSWHHLVNAIGAYIVRNTNLSLEDLIGKDNPDGTHVVGLWELVFQRLVEKGAE
DPSDWLNRRNTIKDVYEKHIAGKPLGTRAYLKKYLPVSDEEVVEILMAARRAL
LPFLKGVKRAGSSGFGVFPYKDTAPRSWNEVEAERKRATGLWYVWSLAFSTA
EYIFTDELSKAGTFFILVKEGKSIKSVFNETLFRSFIEEGLGYKYGMPVRRDELFE
RLVEVFNITDEEVRGVYIDAALSLLSPVGMRTPPCIEEFIMEFAANGNLPEDKVR
HLARWIKLYAKPLKHSTTTTKLVGAGYNVDMRMAVWAKLVEFFAEDDEVAG
ELVRIFKEEYKEAEPPFTCIGARTCPFYLKDDVAKMCPFIFPKEKEILAVSLIDVQ
RHESDGIVILVGGAREVRTFIKKGKVEWVKRTKKTEKYPIAEWFIDVFATEYLG
VPPDNLDFDLKDVTGILKSRERVVPSQLNEVEDWYEKFVEWLKRENEVRGVLP
YKKADVHHLFIKDNMIGIPPALAEEFYRYEANIKPSEFREMLEVKLSIHYKKKA
VKLSVGEKKDVRRCYLVSLEWFRKVVGEPNVKDVVMAGDIALSGLVYYESGE
EVVE

The amino acid sequence of the wild-type primase domain of the Thermococcus celericrescens DNA primase (herein termed “PolpTcele_{Δ295-913}”) is as set forth in SEQ ID NO: 23.

SEQ ID NO: 23-PolpTcele_{Δ295-913}

MSELTPGKVLADVYKVIQDHPEAGRLAIELRFYPVIKSEWVLLNDIEDK
ARDIDKVLAKKNIINGKEAYVSMAIHDFGAVKKKLEKLREKAEGERARR
IGLENVQGEAKGKVHPTVSNYTLALVVDIDIEEVHKSRVVEDVEAVFER
AKKGWLALRPVFEELGVLPRYVFFTGGGLQIWFVAPELEDIAVIDRASG
IVPGVLNALLPEGFVVDNIFDRARIVRAPLTVNHKYKAPNGAGLGVKGR
LIEFNDVRVSLSEVLDKLEAYAKEKGIQLGGQERASGVRVFGKVRYEVK
K

The skilled artisan will be able to readily selected other primase domain from archaeal DNA primases belonging to the primase-polymerase (prim-pol) family of the archaeo-eukaryotic primase (AEP) superfamily, and to identify positionally equivalent amino acid positions to be mutated therein to obtain the desired effect, i.e., a template-independent terminal nucleotidyl transferase activity at least equivalent or improved compared to the corresponding wild-type primase domain.

According to the invention, the functionally active primase domain comprises at least one amino acid substitution at a position positionally equivalent to S68, F74, Y122, F174, N206, N217, F219, P228, L229, T230, 1231, N232, Y233, K234, Y235, K236, and/or P238 in the primase domain of the wild-type Pyrococcus sp. 12-1 DNA primase with SEQ ID NO: 2; or at a position positionally equivalent to S68, F74, Y116, F168, N200, N211, F213, P222, L223, T224, 1225, N226, Y227, K228, Y229, K230, and/or P232 in the primase domain of the wild-type Pyrococcus sp. 12-1 DNA primase with SEQ ID NO: 3.

In some embodiments, the mutated primase domain retains at least 50%, 60%, 70%, 80%, 90%, 95% or 100% of the template-independent terminal nucleotidyl transferase activity of the primase domain with SEQ ID NO: 2 or SEQ ID NO: 3.

Therefore, in a preferred embodiment, the functionally active mutated primase domain is characterized in that:
the mutated primase domain consists of the N-terminal domain of a Pyrococcus sp. 12-1 archaeal DNA primase belonging to the primase-polymerase family and shares at least 80% of sequence identity with the primase domain of SEQ ID NO: 2 or SEQ ID NO: 3;
the mutated primase domain comprises at least one amino acid substitution at position N217, 234, N206, L229, Y233, 236, T230, Y122, F74, F174, F219,81231, P238, Y235, P228, S68, and/or N232 in the amino acid sequence of SEQ ID NO: 2, or at equivalent positions in SEQ ID NO: 3 as determined by sequence and/or structural alignment; and
the mutated primase domain retains at least 50% of the template-independent terminal nucleotidyl transferase activity of the primase domain with SEQ ID NO: 2 or SEQ ID NO: 3.

As a mailer of illustration and convenience, the equivalent positions in the primase domain of the archaeal DNA primase from Thermococcus nautili sp. 30-1, Thermococcus sp. CIR10, Thermococcus peptonophilus, and Thermococcus celericrescens, are as follows:

| PolpP12A_{Δ297-898} | PolpTN2_{Δ311-923} | PolpCIR10_{Δ303-928} | PolpTpep_{Δ295-914} | PolpTcele_{Δ295-913} |
|---|---|---|---|---|
| S68 | S72 | S74 | S71 | S71 |
| F74 | F78 | F80 | F77 | F77 |
| Y122 | F121 | Y128 | Y119 | Y119 |
| F174 | Y173 | F180 | F171 | F171 |
| N206 | N205 | N212 | N203 | N203 |
| N217 | N222 | N223 | N214 | N214 |
| F219 | F224 | F225 | F216 | F216 |
| P228 | P233 | P234 | P225 | P225 |
| L229 | L234 | F235 | L226 | L226 |
| T230 | T235 | T236 | T227 | T227 |
| I231 | F236 | V237 | V228 | V228 |
| N232 | N237 | N238 | N229 | N229 |
| Y233 | H238 | Y239 | H230 | H230 |
| K234 | K239 | K240 | K231 | K231 |
| Y235 | Y240 | Y241 | Y232 | Y232 |
| K236 | R241 | K242 | K233 | K233 |
| P238 | I243 | P244 | P235 | P235 |

In one embodiment, the at least one amino acid substitution comprises:
a) a substitution of the positionally equivalent S68 with an amino acid residue comprising a polar uncharged side chain, such as an asparagine (N), glutamine (Q), or threonine (T) residue; preferably with an asparagine (N) residue;
b) a substitution of the positionally equivalent F74 with an amino acid residue comprising an aromatic side chain, such as a tyrosine (Y), tryptophane (W), or histidine (H) residue, or an amine-containing side chain, such as a glutamine (Q), asparagine (N), arginine (R) or lysine (K) residue; preferably with a tyrosine (Y), or glutamine (Q) residue; more preferably with a tyrosine (Y) residue;
c) a substitution of the positionally equivalent Y122 with an amino acid residue comprising an aromatic side chain, such as a histidine (H), phenylalanine (F), or tryptophan (W) residue, or a hydrophobic side chain such as an alanine (A), glycine (G), valine (V), isoleucine (I), leucine (L), or methionine (M) residue; preferably with an alanine (A), or histidine (H) residue; more preferably with a histidine (H) residue;
d) a substitution of the positionally equivalent F174 with an amino acid residue comprising an amine-containing side chain, such as a arginine (R), glutamine (Q), asparagine (N), or lysine (K) residue, or an amino acid residue comprising an aromatic side chain, such as a tyrosine (Y), tryptophane (W), or histidine (H) residue; preferably with an arginine (R) residue;
e) a substitution of the positionally equivalent N206 with an amino acid residue comprising a positively charged side chain such as an arginine (R), histidine (H), or lysine (K) residue; preferably with an arginine (R) residue;
f) a substitution of the positionally equivalent N217 with an amino acid residue comprising a positively charged side chain such as a lysine (K), arginine (R), or histidine (H) residue; preferably with a lysine (K), or arginine (R) residue; more preferably with a lysine (K) residue;
g) a substitution of the positionally equivalent F219 with an amino acid residue comprising an aromatic side chain, such as a tyrosine (Y), histidine (H), or tryptophan (W) residue; preferably with a tyrosine (Y) residue;
h) a substitution of the positionally equivalent P228 with an amino acid residue comprising a hydrophobic side chain, such as an alanine (A), glycine (G), valine (V), isoleucine (I), leucine (L), or methionine (M) residue, or a polar uncharged side chain, such as an asparagine (N), glutamine (Q), threonine (T) or serine (S) residue; preferably with an alanine (A) or asparagine (N) residue;
i) a substitution of the positionally equivalent L229 with an amino acid residue comprising an amine-containing side chain, such as an asparagine (N), arginine (R), glutamine (Q), or lysine (K) residue, or a hydrophobic side chain such as an alanine (A), glycine (G), valine (V), isoleucine (I), or methionine (M) residue; preferably with an asparagine (N), alanine (A), glycine (G), or arginine (R) residue; more preferably with an asparagine (N) or alanine (A) residue;
j) a substitution of the positionally equivalent T230 with a cysteine (C) residue, or an amino acid residue comprising a hydrophobic side chain such as an alanine (A), glycine (G), valine (V), isoleucine (I), leucine (L), or methionine (M) residue, or a polar uncharged side chain, such as a serine (S), asparagine (N), glutamine (Q), or threonine (T) residue; preferably with a cysteine (C), alanine (A) or serine (S) residue; more preferably with a cysteine (C) residue;
k) a substitution of the positionally equivalent 1231 with an amino acid residue comprising a hydrophobic side chain such as an alanine (A), glycine (G), valine (V), leucine (L), or methionine (M) residue, or a positively charged side chain such as a lysine (K), arginine (R), or histidine (H) residue; preferably with an alanine (A), lysine (K) or arginine (R) residue;
l) a substitution of the positionally equivalent N232 with an amino acid residue comprising an amine-containing side chain, such as an arginine (R), asparagine (N), glutamine (Q), lysine (K) or histidine (H) residue; preferably with an arginine (R) residue;
m) a substitution of the positionally equivalent Y233 with an amino acid residue comprising a positively charged side chain such as a lysine (K), histidine (H) or arginine (R) residue, or a hydrophobic side chain such as an alanine (A), glycine (G), valine (V), isoleucine (I), leucine (L), or methionine (M) residue; preferably with a lysine (K), histidine (H), arginine (R), or alanine (A) residue; more preferably with a lysine (K) or histidine (H) residue;
n) a substitution of the positionally equivalent K234 with an amino acid residue comprising a positively charged side chain such as an arginine (R), or histidine (H) residue; preferably with an arginine (R) residue;
o) a substitution of the positionally equivalent Y235 with an amino acid residue comprising an aromatic side chain, such as a phenylalanine (F), tryptophan (W), or histidine (H) residue; preferably with a phenylalanine (F) or tryptophan (W) residue; more preferably with a phenylalanine (F) residue;
p) a substitution of the positionally equivalent K236 with an amino acid residue comprising a positively charged side chain such as an arginine (R), or histidine (H) residue; preferably with an arginine (R) residue; and/or
q) a substitution of the positionally equivalent P238 with an amino acid residue comprising a positively charged side chain such as an arginine (R), histidine (H), or lysine (K) residue; preferably with an arginine (R) residue;
   in the primase domain of the wild-type Pyrococcus sp. 12-1 DNA primase with SEQ ID NO: 2.

In one embodiment, the at least one amino acid substitution is positionally equivalent to S68N, F74Y, F74Q, Y122A, Y122H, F174R, N206R, N217K, N217R, F219Y, P228N, P228A, L229A, L229G, L229N, L229R, T230A, T230C, T230S, I231A, 1231R, 1231K, N232R, Y233A, Y233H, Y233R, Y233K, K234R, Y235F, Y235W, K236R, and/or P238R in the primase domain of the wild-type Pyrococcus sp. 12-1 DNA primase with SEQ ID NO: 2; or positionally equivalent to S68N, F74Y, F74Q, Y116A, Y116H, F168R, N200R, N211K, N211R, F213Y, P222N, P222A, L223A, L223G, L223N, L223R, T224A, T224C, T224S, I225A, I225R, I225K, N226R, Y227A, Y227H, Y227R, Y227K, K228R, Y229F, Y229W, K230R and/or P232R in the primase domain of the wild-type Pyrococcus sp. 12-1 DNA primase with SEQ ID NO: 3.

In one embodiment, the functionally active primase domain comprises at least one amino acid substitution at a position positionally equivalent to F74, Y122, F174, N206, N217, F219, L229, T230, 1231, Y233, K234, K236, and/or P238 in the primase domain of the wild-type Pyrococcus sp. 12-1 DNA primase with SEQ ID NO: 2; or at a position positionally equivalent to F74, Y116, F168, N200, N211, F213, L223, T224, 1225, Y227, K228, K230, and/or P232in the primase domain of the wild-type Pyrococcus sp. 12-1 DNA primase with SEQ ID NO: 3.

In one embodiment, the at least one amino acid substitution is positionally equivalent to F74Y, Y122A, Y122H, F174R, N206R, N217K, F219Y, L229A, L229G, L229N, L229R, T230A, T230C, T230S, 1231A, 1231R, 1231K, Y233A, Y233H, Y233R, Y233K, K234R, K236R and/or P238R in the primase domain of the wild-type Pyrococcus sp. 12-1 DNA primase with SEQ ID NO: 2; or positionally equivalent to F74Y, Y116A, Y116H, F168R, N200R, N211K, F213Y, L223A, L223G, L223N, L223R, T224A, T224C, T224S, I225A, I225R, I225K, Y227A, Y227H, Y227R, Y227K, K228R, K230R, and/or P232Rin the primase domain of the wild-type Pyrococcus sp. 12-1 DNA primase with SEQ ID NO: 3.

In one embodiment, the functionally active primase domain comprises at least one amino acid substitution at a position positionally equivalent to F74, Y122, N206, N217, L229, T230, Y233, K234, and/or K236 in the primase domain of the wild-type Pyrococcus sp. 12-1 DNA primase with SEQ ID NO: 2; or at a position positionally equivalent to F74, Y116, N200, N211, L223, T224, Y227, K228, and/or K230 in the primase domain of the wild-type Pyrococcus sp. 12-1 DNA primase with SEQ ID NO: 3.

In one embodiment, the at least one amino acid substitution is positionally equivalent to F74Y, Y122H, N206R, N217K, L229A, L229N, T230C, Y233H, Y233K, K234R, and/or K236R in the primase domain of the wild-type Pyrococcus sp. 12-1 DNA primase with SEQ ID NO: 2; or positionally equivalent to F74Y, Y116H, N200R, N211K, L223A, T224C, Y227H, and/or K230R in the primase domain of the wild-type Pyrococcus sp. 12-1 DNA primase with SEQ ID NO: 3.

In one embodiment, the functionally active primase domain comprises more than one amino acid substitution as defined above, such as, e.g., two, three, four, five or more amino acid substitutions.

In one embodiment, the functionally active primase domain comprises two amino acid substitutions at positions positionally equivalent to F74, Y122, N206, N217, T230, Y233, K234 and/or K236 in the primase domain of the wild-type Pyrococcus sp. 12-1 DNA primase with SEQ ID NO: 2; or at positions positionally equivalent to F74, Y116, N200, N211, T224, Y227, K228 and/or K230 in the primase domain of the wild-type Pyrococcus sp. 12-1 DNA primase with SEQ ID NO: 3.

In one embodiment, the two one amino acid substitutions are positionally equivalent to F74Y, Y122H, N206R, N217K, T230C, Y233H, K234R and/or K236R in the primase domain of the wild-type Pyrococcus sp. 12-1 DNA primase with SEQ ID NO: 2; or positionally equivalent to F74Y, Y116H, N200R, N211K, T224C, Y227H, K228R and/or K230R in the primase domain of the wild-type Pyrococcus sp. 12-1 DNA primase with SEQ ID NO: 3.

In one embodiment, the functionally active primase domain comprises two amino acid substitutions at positions positionally equivalent to N217 and K234; N217 and K236; N217 and N206; Y122 and Y233; Y122 and N217; Y122 and K234; Y122 and K236; Y122 and N206; Y122 and T230; F74 and N217; F74 and K234; K234 and T230; K236 and T230; N206 and Y233; N206 and T230; or T230 and N217 in the primase domain of the wild-type Pyrococcus sp. 12-1 DNA primase with SEQ ID NO: 2; or at positions positionally equivalent to N211 and K228; N211 and K230; N211 and N200; Y116 and Y227; Y116 and N211; Y116 and K228; Y116 and K230; Y116 and N200; Y116 and T224; F74 and N211; F74 and K228; K228 and T224; K230 and T224; N200 and Y227; N200 and T224; or T224 and N211 in the primase domain of the wild-type Pyrococcus sp. 12-1 DNA primase with SEQ ID NO: 3.

In one embodiment, the functionally active primase domain comprises two amino acid substitutions at positions positionally equivalent to N217K and K234R; N217K and K236R; N217K and N206R; Y122H and Y233H; Y122H and N217K; Y122H and K234R; Y122H and K236R; Y122H and N206R; Y122H and T230C; F74Y and N217K; F74Y and K234R; K234R and T230C; K236R and T230C; N206R and Y233H; N206R and T230C; or T230C and N217K in the primase domain of the wild-type Pyrococcus sp. 12-1 DNA primase with SEQ ID NO: 2; or at positions positionally equivalent to N211K and K228R; N211K and K230R; N2111K and N200R; Y116H and Y227H; Y116H and N2111K; Y116H and K228R; Y116H and K230R; Y116H and N200R; Y116H and T224C; F74Y and N211K; F74Y and K228R; K228R and T224C; K230R and T224C; N200R and Y227H; N200R and T224C; or T224C and N211K in the primase domain of the wild-type Pyrococcus sp. 12-1 DNA primase with SEQ ID NO: 3.

In one embodiment, the functionally active primase domain comprises two amino acid substitutions at positions positionally equivalent to N206 and Y233; N206 and T230; Y122 and N206; N217 and N206; Y122 and N217; or Y122 and Y233 in the primase domain of the wild-type Pyrococcus sp. 12-1 DNA primase with SEQ ID NO: 2; or at positions positionally equivalent to N200 and Y227; N200 and T224; Y116 and N200; N211 and N200; Y116 and N211; or Y116 and Y227 in the primase domain of the wild-type Pyrococcus sp. 12-1 DNA primase with SEQ ID NO: 3.

In one embodiment, the functionally active primase domain comprises two amino acid substitutions at positions positionally equivalent to N206R and Y233H; N206R and T230C; Y122H and N206R; N217K and N206R; Y122H and N217K; or Y122H and Y233H in the primase domain of the wild-type Pyrococcus sp. 12-1 DNA primase with SEQ ID NO: 2; or at positions positionally equivalent to N200R and Y227H; N200R and T224C; Y 116H and N200R; N211K and N200R; Y 116H and N211K; Y116H and Y227H in the primase domain of the wild-type Pyrococcus sp. 12-1 DNA primase with SEQ ID NO: 3.

In one embodiment, the functionally active primase domain comprises three amino acid substitutions at positions positionally equivalent to Y122, N206, N217, Y233, and/or K234 in the primase domain of the wild-type Pyrococcus sp. 12-1 DNA primase with SEQ ID NO: 2; or at positions positionally equivalent to Y116, N200, N211, Y227, and/or K228 in the primase domain of the wild-type Pyrococcus sp. 12-1 DNA primase with SEQ ID NO: 3.

In one embodiment, the three one amino acid substitutions are positionally equivalent to Y122H, N206R, N217K, Y233H, and/or K234R in the primase domain of the wild-type Pyrococcus sp. 12-1 DNA primase with SEQ ID NO: 2; or positionally equivalent to Y116H, N200R, N211K, Y227H, and/or K228R in the primase domain of the wild-type Pyrococcus sp. 12-1 DNA primase with SEQ ID NO: 3.

In one embodiment, the functionally active primase domain comprises three amino acid substitutions at positions positionally equivalent to N206, N217, and Y233 in the primase domain of the wild-type Pyrococcus sp. 12-1 DNA primase with SEQ ID NO: 2; or at positions positionally equivalent to N200, N211, and Y227 in the primase domain of the wild-type Pyrococcus sp. 12-1 DNA primase with SEQ ID NO: 3.

In one embodiment, the three one amino acid substitutions are positionally equivalent to N206R, N217K, and Y233H in the primase domain of the wild-type Pyrococcus sp. 12-1 DNA primase with SEQ ID NO: 2; or positionally equivalent to N200R, N211K, and Y227H in the primase domain of the wild-type Pyrococcus sp. 12-1 DNA primase with SEQ ID NO: 3. An exemplary amino acid sequence of functionally active primase domain comprising these three substitutions is as set forth in SEQ ID NO: 24 (herein termed “SHEE-4.3/13.1/3.24”).

SEQ ID NO: 24-SHEE-4.3/13.1/3.24 (mutated residues in bold)

MRPSDIIIDVYKAIQDHPGAGKLAIELRFYPRPTSEWIIVADIEDKAEELHKVLFK
NNVLGKKEAYISMALHDFEEVGKKLEKLRELEEERAQKEGRKPREVTLRNVQG
EATGKVHKTVSKYTLTLVVDIDVEEIHKSKVVESEEKAFELAKRAWDELKPKL
EGIGVKPRYVFFTGGGVQLWFVAPGLEPIEVIDRASRVIPPVLRAMLPEGYSVD
KIFDRARIVRVPLTINHKYKTPDERPLEIRGRLIEFNDVRTPLGEVLDKLEAYAK
EHGISLVTPSQARFIGTVGRYEVDK

In one embodiment, the functionally active primase domain comprises three amino acid substitutions at positions positionally equivalent to Y122, N206, and N217 in the primase domain of the wild-type Pyrococcus sp. 12-1 DNA primase with SEQ ID NO: 2; or at positions positionally equivalent to Y116, N200, and N211 in the primase domain of the wild-type Pyrococcus sp. 12-1 DNA primase with SEQ ID NO: 3.

In one embodiment, the three one amino acid substitutions are positionally equivalent to Y122H, N206R, and N217K in the primase domain of the wild-type Pyrococcus sp. 12-1 DNA primase with SEQ ID NO: 2; or positionally equivalent to Y116H, N200R, and N211K in the primase domain of the wild-type Pyrococcus sp. 12-1 DNA primase with SEQ ID NO: 3. An exemplary amino acid sequence of functionally active primase domain comprising these three substitutions is as set forth in SEQ ID NO: 25 (herein termed “SHEE-4.3/13.1/9.2”).

SEQ ID NO: 25-SHEE-4.3/13.1/9.2 (mutated residues in bold)

MRPSDIIIDVYKAIQDHPGAGKLAIELRFYPRPTSEWIIVADIEDKAEELHKVLFK
NNVLGKKEAYISMALHDFEEVGKKLEKLRELEEERAQKEGRKPREVTLRNVQG
EATGKVHKTVSKHTLTLVVDIDVEEIHKSKVVESEEKAFELAKRAWDELKPKL
EGIGVKPRYVFFTGGGVQLWFVAPGLEPIEVIDRASRVIPPVLRAMLPEGYSVD
KIFDRARIVRVPLTINYKYKTPDERPLEIRGRLIEFNDVRTPLGEVLDKLEAYAK
EHGISLVTPSQARFIGTVGRYEVDK

In one embodiment, the functionally active primase domain comprises three amino acid substitutions at positions positionally equivalent to N206, N217, and K234 in the primase domain of the wild-type Pyrococcus sp. 12-1 DNA primase with SEQ ID NO: 2; or at positions positionally equivalent to N200, N211, and K228 in the primase domain of the wild-type Pyrococcus sp. 12-1 DNA primase with SEQ ID NO: 3.

In one embodiment, the three one amino acid substitutions are positionally equivalent to N206R, N217K, and K234R in the primase domain of the wild-type Pyrococcus sp. 12-1 DNA primase with SEQ ID NO: 2; or positionally equivalent to N200R, N211K, and K228R in the primase domain of the wild-type Pyrococcus sp. 12-1 DNA primase with SEQ ID NO: 3. An exemplary amino acid sequence of functionally active primase domain comprising these three substitutions is as set forth in SEQ ID NO: 26 (herein termed “SHEE-4.3/13.1/11.1”).

SEQ ID NO: 26-SHEE-4.3/13.1/11.1 (mutated residues in bold)

MRPSDIIIDVYKAIQDHPGAGKLAIELRFYPRPTSEWIIVADIEDKAEELHKVLFK
NNVLGKKEAYISMALHDFEEVGKKLEKLRELEEERAQKEGRKPREVTLRNVQG
EATGKVHKTVSKYTLTLVVDIDVEEIHKSKVVESEEKAFELAKRAWDELKPKL
EGIGVKPRYVFFTGGGVQLWFVAPGLEPIEVIDRASRVIPPVLRAMLPEGYSVD
KIFDRARIVRVPLTINYRYKTPDERPLEIRGRLIEFNDVRTPLGEVLDKLEAYAK
EHGISLVTPSQARFIGTVGRYEVDK

In another embodiment, the functionally active primase domain is the primase domain of the archaeal DNA primase from Thermococcus nautili sp. 30-1.

In one embodiment, the functionally active primase domain shares at least 50%, 55%, 60%, 65%, 70%, 75%, 80%, 85%, 90%, 95%, 96%, 97%, 98%, 99%, 99.5%, 99.9% or 100% of sequence identity with the primase domain of from Thermococcus nautili sp. 30-1 with SEQ ID NO: 5.

In one embodiment, the functionally active primase domain shares at least 50%, 55%, 60%, 65%, 70%, 75%, 80%, 99%, 99.1%, 99.2%, 99.3%, 99.4%, 99.5%, 99.6%, 99.7%, 99.8%, 99.9% or 100% of sequence identity with the primase domain of from Thermococcus nautili sp. 30-1 with SEQ ID NO: 5.

In one embodiment, the functionally active primase domain is the primase domain of the archaeal DNA primase from Thermococcus nautili sp. 30-1, with SEQ ID NO: 5.

In another embodiment, the functionally active mutated primase domain is characterized in that:
the mutated primase domain consists of the N-terminal domain of a Thermococcus nautili sp. 30-1 archaeal DNA primase belonging to the primase-polymerase family and shares at least 80% of sequence identity with the primase domain of SEQ ID NO: 5;
the mutated primase domain comprises at least one amino acid substitution at position F121, N205, N222 and/or K239 in the amino acid sequence of SEQ ID NO: 5, or at the equivalent positions in the primase domain of the archaeal DNA primase from Thermococcus sp. CIR10, Thermococcus peptonophilus, and Thermococcus celericrescens as determined by sequence and/or structural alignment; and
the mutated primase domain retains at least 50% of the template-independent terminal nucleotidyl transferase activity of the primase domain with SEQ ID NO: 5.

The amino acid sequence of the wild-type primase domain of the Thermococcus nautili sp. 30-1 DNA primase (herein termed “PolpTN2_{Δ311-923}”) is as set forth in SEQ ID NO: 5.

In one embodiment, the wild-type primase domain of the archaeal DNA primase from Thermococcus nautili sp. 30-1 has an amino acid sequence with any one of SEQ ID NOs: 5-16 or 30-32. In one embodiment, the wild-type primase domain of the archaeal DNA primase from Thermococcus nautili sp. 30-1 has an amino acid sequence with any one of SEQ ID NOs: 5-16. In one embodiment, the wild-type primase domain of the archaeal DNA primase from Thermococcus nautili sp. 30-1 has an amino acid sequence with any one of SEQ ID NOs: 30-32.

In one embodiment, the wild-type primase domain of the archaeal DNA primase from Thermococcus nautili sp. 30-1 has an amino acid sequence with any one of SEQ ID NOs: 30-31.

In one embodiment, the mutated primase domain comprises at least one amino acid substitution at position F121H, N205R, N222K, and/or K239R in the amino acid sequence of SEQ ID NO: 5.

In one embodiment, the mutated primase domain comprises amino acid substitutions F121H, N205R and N222K with reference to SEQ ID NO: 5 numbering, as set forth in SEQ ID NO: 30.

SEQ ID NO: 30-PolpTN2Δ₃₁₁₋₉₂₃_B

MSSLRPSSIIIDIYKVIQDHPDSSRWAIELRFLPKPISSEWIFITDIEERASEIDKVLT
KYNIMKKKDAYVSMAIHDFEKVTAKLKRVQEEENKKATEGERRLRRITLDKIQ
GDSETTSGHTLALVVDIDNTKIHDTRIIENEEEAFEASKREWEALKPKLQELGFL
PRWILYTGGGLQLWFVSDKLEPISVIDRASEIIPNIMRGVNGVKGLLSEGFKADK
IFDPARIVRAPLTFNHKYRTIIKDEDGTERVVPTQVKGRVIEFNDVRISLTEFLDR
LEAYAKEKGIPLEKPTKRKFLELASKRYEVTS

In one embodiment, the mutated primase domain comprises amino acid substitutions N205R, N222K and K239R with reference to SEQ ID NO: 5 numbering, as set forth in SEQ ID NO: 31.

In this embodiment, the mutated primase domain decreases the ab-initio single-stranded nucleic acid synthesis activity as compared to the ab-initio single-stranded nucleic acid synthesis activity of the archaeal DNA primase from Thermococcus nautili sp. 30-1 with an amino acid sequence with any one of SEQ ID NOs: 5-16.

SEQ ID NO: 31-PolpTN2Δ₃₁₁₋₉₂₃_C

MSSLRPSSIIIDIYKVIQDHPDSSRWAIELRFLPKPISSEWIFITDIEERASEIDKVLT
KYNIMKKKDAYVSMAIHDFEKVTAKLKRVQEEENKKATEGERRLRRITLDKIQ
GDSETTSGFTLALVVDIDNTKIHDTRIIENEEEAFEASKREWEALKPKLQELGFL
PRWILYTGGGLQLWFVSDKLEPISVIDRASEIIPNIMRGVNGVKGLLSEGFKADK
IFDPARIVRAPLTFNHRYRTIIKDEDGTERVVPTQVKGRVIEFNDVRISLTEFLDR
LEAYAKEKGIPLEKPTKRKFLELASKRYEVTS

In one embodiment, the mutated primase domain retains at least 50%, 60%, 70%, 80%, 90%, 95% or 100% of the ab-initio activity of the primase domain with SEQ ID NO: 5.

In another embodiment, the mutated primase domain comprises amino acid substitutions F121H, N205R, N222K and K239R with reference to SEQ ID NO: 5 numbering, as set forth in SEQ ID NO: 32. In one embodiment, the wild-type primase domain of the archaeal DNA primase from Thermococcus nautili sp. 30-1 has an amino acid sequence of SEQ ID NO: 32.

In this embodiment, the mutated primase domain sensibly abolishes the ab-initio single-stranded nucleic acid synthesis activity as compared to the ab-initio single-stranded nucleic acid synthesis activity of the archaeal DNA primase from Thermococcus nautili sp. 30-1 with an amino acid sequence with any one of SEQ ID NOs: 5-16.

SEQ ID NO: 32-PolpTN2Δ₃₁₁₋₉₂₃_D

MSSLRPSSIIIDIYKVIQDHPDSSRWAIELRFLPKPISSEWIFITDIEERASEIDKVLT
KYNIMKKKDAYVSMAIHDFEKVTAKLKRVQEEENKKATEGERRLRRITLDKIQ
GDSETTSGHTLALVVDIDNTKIHDTRIIENEEEAFEASKREWEALKPKLQELGFL
PRWILYTGGGLQLWFVSDKLEPISVIDRASEIIPNIMRGVNGVKGLLSEGFKADK
IFDPARIVRAPLTFNHRYRTIIKDEDGTERVVPTQVKGRVIEFNDVRISLTEFLDR
LEAYAKEKGIPLEKPTKRKFLELASKRYEVTS

In this embodiment, the mutated primase domain sensibly avoids the ab-initio single-stranded nucleic acid synthesis activity, i.e., the mutated primase domain is devoid of ab-initio single-stranded nucleic acid synthesis activity.

In this embodiment, the mutated primase domain sensibly avoids the ab-initio single-stranded nucleic acid synthesis activity as compared to the ab-initio single-stranded nucleic acid synthesis activity of the archaeal DNA primase from Thermococcus nautili sp. 30-1 with an amino acid sequence with any one of SEQ ID NOs: 5-16.

According to the invention, the mutated primase domain is functionally active.

According to the invention, the functionally active mutated primase domain is capable of template-independent terminal nucleotidyl transferase activity.

According to the invention, the functionally active mutated primase domain retains at least 50% of the template-independent terminal nucleotidyl transferase activity of its corresponding wild-type primase domain.

In some embodiments, the mutated primase domain retains at least 50%, 60%, 70%, 80%, 90%, 95% or 100% of the template-independent terminal nucleotidyl transferase activity of the primase domain with SEQ ID NO: 5.

In one embodiment, the functionally active mutated primase domain has at least an equivalent template-independent terminal nucleotidyl transferase activity compared to its corresponding wild-type primase domain. By “equivalent activity”, it is meant that the relative template-independent terminal nucleotidyl transferase activity of both the wild-type primase domain and the mutated primase domain are within ±5%, ±10%, ±15%, ±20%, ±25% or ±30%.

In one embodiment, the functionally active mutated primase domain has improved template-independent terminal nucleotidyl transferase activity compared to its corresponding wild-type primase domain. By “improved”, it is meant that the relative template-independent terminal nucleotidyl transferase activity of the mutated primase domain is at least 20% higher, preferably at least 30%, 40%, 50%, 60%, 70%, 80%, 90%, 100%, 150%, 200%, 250% or more higher than the relative template-independent terminal nucleotidyl transferase activity of its corresponding wild-type primase domain.

In one embodiment, the functionally active mutated primase domain has reduced ab-initio single-stranded nucleic acid synthesis activity compared to its corresponding wild-type primase domain. By “reduced”, it is meant that the relative ab-initio single-stranded nucleic acid synthesis activity of the mutated primase domain is at least 20% lower, preferably at least 30%, 40%, 50%, 60%, 70%, 80%, 90% or more lower than the relative ab-initio single-stranded nucleic acid synthesis activity of its corresponding wild-type primase domain. In one embodiment, the mutated primase domain is devoid of ab-initio single-stranded nucleic acid synthesis activity.

In one embodiment, the mutated primase domain is isolated.

“Isolated” and any declensions thereof, as well as “purified” and any declensions thereof, are used interchangeably and mean that the mutated primase domain is substantially free of other components (i.e., of contaminants). Preferably, an isolated (or purified) mutated primase domain is substantially free of other proteins or nucleic acids with which it is associated in a cell, such as, e.g., in a recombinant expression cell system. By “substantially free”, it is meant that the isolated (or purified) mutated primase domain represents more than 50% of a heterogeneous composition (i.e., is at least 50% pure), preferably, more than 60%, more than 70%, more than 80%, more than 90%, more than 95%, and more preferably still more than 98% or 99%. Purity can be assessed by various methods known by the skilled artisan, including, but not limited to, chromatography, gel electrophoresis, immunoassay, composition analysis, biological assay, and the like.

In one embodiment, the mutated, functionally active primase domain may be fused to a processivity factor.

By “processivity factor”, it is meant a polypeptide domain or subdomain which confers sequence-independent nucleic acid interactions, and is associated with the mutated, functionally active primase domain by covalent or noncovalent interaction(s). Processivity factors may confer a lower dissociation constant between the primase domain and the nucleic acid substrate, allowing for more nucleotide incorporations on average before dissociation of the primase domain from the substrate or initiator sequence.

Processivity factors function by multiple sequence-independent nucleic acid binding mechanisms: the primary mechanism is electrostatic interaction between the nucleic acid phosphate backbone and the processivity factor; the second is steric interactions between the processivity factor and the minor groove structure of a nucleic acid duplex; the third mechanism is topological restraint, where interactions with the nucleic acid are facilitated by clamp proteins that completely encircle the nucleic acid, with which they associate.

Exemplary sequence-independent nucleic acid binding domains are known in the art, and are traditionally classified according to the preferred nucleic acid substrate, e.g., DNA or RNA and strandedness, such as single-stranded or double-stranded.

Various polypeptide domains have been identified as nucleic acid binders. These polypeptide domains include four general structural topologies known to bind single-stranded DNA: oligonucleotide-binding (OB) folds, K homology (KH) domains, RNA recognition motifs (RRMs), and whirly domains, as described in Dickey et al., 2013. Structure. 21(7):1074-1084.

Oligonucleotide-binding domains (OBDs) are exemplary DNA binding domains structurally conserved in multiple DNA processing proteins. OBDs bind with single-stranded DNA ligands from 3 to 11 nucleotides per OB fold and dissociation constants ranging from low-picomolar to high-micromolar levels. Affinities roughly correlate with the length of single-stranded DNA bound. Some OBDs may confer sequence specific binding, while others are non-sequence specific. Exemplary OBD containing DNA-binding proteins specifically bind single-stranded DNA are so called “single-stranded DNA binding proteins” or “SSBs”. SSB domains are well known to those skilled in the art, as described, e.g., in Keck (Ed.), 2016. Single-stranded DNA binding proteins (Vol. 922, Methods in Molecular Biology). Totowa, NJ: Humana Press; and Shereda et al., 2008. Crit Rev Biochem Mol Biol. 43(5):289-318. SSBs describe a family of evolved molecular chaperones of single-stranded DNA.

Several exemplary prokaryotic SSBs have been characterized as known to those skilled in the art. These SSBs include, but are not limited to; Escherichia coli SSB (see, e.g., Raghunathan et al., 2000. Nat Struct Biol. 7(8):648-652), Deinococcus radiodurans SSB (see, e.g., Lockhart & DeVeaux, 2013. PLoS One. 8(8):e71651), Sulfolobus solfataricus SSB (see, e.g., Paytubi et al., 2012. Proc Natl Acad Sci USA. 109(7):E398-E405), Thermus thermophillus SSB and Thermus aquaticus SSB (see, e.g., Witte et al., 2008. Biophys J. 94(6):2269-2279), and Deinococcus radiopugnans SSB (see, e.g., Filipkowski et al., 2006. Extremophiles. 10(6):607-614).

In non-eubacterial systems, functional eukaryotic homologs to the prokaryotic SSB protein family are known to those skilled in the art. Replication protein A (RPA) is an exemplary homolog used in DNA replication, recombination and DNA repair in eukaryotes. The RPA heterotrimer is comprised of RPA70, RPA32, RPA14 subunits as described in Iftode et al., 1999. Crit Rev Biochem Mol Biol. 34(3):141-180.

In one embodiment, the oligonucleotide binding domain is fused in N-terminal of the mutated, functionally active primase domain, optionally through a linker. In one embodiment, the oligonucleotide binding domain is fused in C-terminal of the mutated, functionally active primase domain, optionally through a linker.

An exemplary oligonucleotide binding domain is the single stranded DNA-binding protein (ssb) from Thermotoga neapolitana.

The amino acid sequence of the Thermotoga neapolitana ssb comprises or consists of SEQ ID NO: 27, which represents the amino acid sequence of the protein “single stranded DNA-binding protein” from Thermotoga neapolitana with NCBI Reference Sequence ACY68958.1 version 1 of 2010-10-25.

SEQ ID NO: 27-Thermotoga neapolitana strain DSMZ 4359 ssb

MSFFNRIILIGRLVRDPEERYTLSGTPVTTFTIAVDRVPRKNAPDDAQTTDFFRV
VTFGRLAEFARTYLTKGRLILVEGEMRMRRWETQTGEKRVSPEVVANVVRFM
DRKPVEMPSEDIEEKLEIPEEDFTDDTFSEDEPPF

The present invention also relates to a nucleic acid encoding the mutated, functionally active primase domain described above.

It also relates to an expression vector comprising the nucleic acid encoding the mutated, functionally active primase domain described above.

It also relates to a host cell comprising the expression vector comprising the nucleic acid encoding the mutated, functionally active primase domain described above.

It also relates to a method of producing and purifying the mutated, functionally active primase domain described above.

In one embodiment, the method comprises:
culturing a host cell comprising the expression vector comprising the nucleic acid encoding the mutated, functionally active primase domain described above, under conditions suitable for the expression of said mutated, functionally active primase domain, and
isolating the mutated, functionally active primase domain from said host cell.

This recombinant process can be used for large scale production of the mutated, functionally active primase domain.

In one embodiment, the expressed mutated, functionally active primase domain is further purified, e.g., by means well known to the skilled artisan. Exemplary processes for purifying the expressed mutated, functionally active primase domain are described in WO2011098588 and Gill et al., 2014 (Nucleic Acids Res. 42(6):3707-3719).

In a second aspect, the present invention relates to a method for template-independent synthesis of nucleic acids, comprising iteratively contacting an initiator sequence comprising a 3′-end nucleotide with a free 3′-hydroxyl group, with at least one (optionally, selected) nucleoside triphosphate (or a combination of (optionally, selected) nucleoside triphosphates) in the presence of a mutated, functionally active primase domain described above, thereby covalently binding said (optionally, selected) nucleoside triphosphate to the free 3′-hydroxyl group of the 3′-end nucleotide.

In one embodiment, the method is for template-independent synthesis of nucleic acids with random nucleotide sequence. In one embodiment, the method is for template-independent, sequence-controlled synthesis of nucleic acids.

References to a “nucleic acid” synthesis method include methods of synthesizing lengths of DNA (deoxyribonucleic acid), RNA (ribonucleic acid), or mixes thereof, wherein a strand of nucleic acid (i.e., an initiator sequence) comprising “n” nucleotides is iteratively extended by adding a further nucleotide “n+1”. The term “nucleic acid” also encompasses nucleic acid analogues, such as, without limitation, xeno nucleic acids (XNA), which are synthetic nucleic acid analogues that have a different sugar backbone and/or outgoing motif than the natural DNAs and RNAs. The term “nucleic acid” hence also encompasses mixed XNA/DNA, mixed XNA/RNA and mixed XNA/DNA/RNA. Examples of XNAs include those described in Schmidt, 2010. Bioessays. 32(4):322-331 and Nie et al., 2020. Molecules. 25(15):E3483, the content of which is herein incorporated by reference. Some examples include, but are not limited to, 1,5-anhydrohexitol nucleic acid (HNA), cyclohexene nucleic acid (CeNA), threose nucleic acid (TNA), glycol nucleic acid (GNA), locked nucleic acid (LNA), peptide nucleic acid (PNA), and fluoro arabino nucleic acid (FANA) (Schmidt, 2008. Syst Synth Biol. 2(1-2):1-6; Ran et al., 2009. Nat Nanotechnol. 4(10):6; Kershner et al., 2009. Nat Nanotechnol. 4(9):557-61; Marliere, 2009. Syst Synth Biol. 3(1-4):77-84; Torres et al., 2003. Microbiology. 149(Pt 12):3595-601; Vastmans et al., 2001. Nucleic Acids Res. 29(15):3154-63; Ichida et al., 2005. Nucleic Acids Res. 33(16):5219-25; Kempeneers et al., 2005. Nucleic Acids Res. 33(12):3828-36; Loakes et al., 2009. J Am Chem Soc. 131(41):14827-37).

References to a “template-independent” nucleic acid synthesis method illustrate those methods of nucleic acid synthesis which do not require a template nucleic acid strand, i.e., the nucleic acid is synthesized de novo.

References to a “sequence-controlled” nucleic acid synthesis method illustrate those methods of nucleic acid synthesis which allow the specific addition of selected nucleotides “n+1” to a strand of nucleic acid (i.e., an initiator sequence) comprising “n” nucleotides, i.e., the synthesized nucleic acid has a defined—by contrast to random-nucleotide sequence.

By “initiator sequence” or “primer”, it is meant a short oligonucleotide with a free 3′-end onto which a (optionally, selected) nucleoside triphosphate can be covalently bound, i.e., the nucleic acid will be synthesized from the 3′-end of the initiator sequence.

In one embodiment, the initiator sequence is a DNA initiator sequence. In one embodiment, the initiator sequence is an RNA initiator sequence. In one embodiment, the initiator sequence is an XNA initiator sequence. In one embodiment, the initiator sequence is a mixed DNA/RNA initiator sequence. In one embodiment, the initiator sequence is a mixed XNA/DNA initiator sequence. In one embodiment, the initiator sequence is a mixed XNA/RNA initiator sequence. In one embodiment, the initiator sequence is a mixed XNA/DNA/RNA initiator sequence.

In one embodiment, the initiator sequence has a length ranging from 2 to 50 nucleotides. In one embodiment, the initiator sequence comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

In one embodiment, the initiator sequence is single-stranded. In one embodiment, the initiator sequence is double-stranded. In the latter embodiment, it will be understood by the one skilled in the art that a 3′-overhang (i.e., a free 3′-end) is preferable for a more efficient binding of the (optionally, selected) nucleoside triphosphate.

In one embodiment, the initiator sequence may be immobilized onto a support. In particular, the use of supports allows to easily filter, wash and/or elute reagents and by-products, without washing away the synthesized nucleic acid.

Suitable examples of supports include, but are not limited to, beads, slides, chips, particles, strands, gels, sheets, tubing, spheres, containers, capillaries, pads, slices, films, culture dishes, microtiter plates, and the like. Exemplary materials that can be used for such supports include, but are not limited to, acrylics, carbon (e.g., graphite, carbon-fiber), cellulose (e.g., cellulose acetate), ceramics, controlled-pore glass, cross-linked polysaccharides (e.g., agarose, SEPHAROSE™ or alginate), gels, glass (e.g., modified or functionalized glass), gold (e.g., atomically smooth Au(111)), graphite, inorganic glasses, inorganic polymers, latex, metal oxides (e.g., SiO₂, TiO₂, stainless steel), metalloids, metals (e.g., atomically smooth Au(111)), mica, molybdenum sulfides, nanomaterials (e.g., highly oriented pyrolitic graphite (HOPG) nanosheets), nitrocellulose, NYLON™, optical fiber bundles, organic polymers, paper, plastics, polacryloylmorpholide, poly(4-methylbutene), polyethylene terephthalate), poly(vinyl butyrate), polybutylene, polydimethylsiloxane (PDMS), polyethylene, polyformaldehyde, polymethacrylate, polypropylene, polysaccharides, polystyrene, polyurethanes, polyvinylidene difluoride (PVDF), quartz, rayon, resins, rubbers, semiconductor material, silica, silicon (e.g., surface-oxidized silicon), sulfide, and TEFLON™; or a mixture thereof.

In one embodiment, the initiator sequence is immobilized onto a support via a reversible interacting moiety, such as, e.g., a chemically-cleavable linker, an enzymatically-cleavable linker, or any other suitable means. It is thus conceivable that the synthetized nucleic acid be ultimately cleaved from the support and, e.g., amplified using the initiator sequence as a template. The initiator sequence could therefore contain an appropriate forward primer sequence, and an appropriate reverse primer could be synthesized.

Additionally or alternatively, the immobilized initiator sequence may contain a restriction site. It is thus conceivable that the synthetized nucleic acid be ultimately cleaved from the support using a restriction enzyme.

Additionally or alternatively, the immobilized initiator sequence may contain a uridine. It is thus conceivable that the synthetized nucleic acid be ultimately cleaved from the support using (1) an uracil-DNA glycosylase (UDG) to generate an abasic site, and (2) an apurinic/apyrimidinic (AP) site endonuclease to cleave the synthetized nucleic acid at the abasic site.

Additionally or alternatively, the immobilized initiator sequence may contain a sequence complementary to a small interfering nucleic acid guide sequence. It is thus conceivable that the synthetized nucleic acid be ultimately cleaved from the support using a small interfering nucleic acid guide sequence to target an endonuclease such as, e.g., Argonaute, to the immobilized initiator sequence and cleave the synthetized nucleic acid.

By “nucleoside triphosphate” or “NTP”, it is referred herein to a molecule containing a nitrogenous base bound to a 5-carbon sugar (typically, either ribose or deoxyribose), with three phosphate groups bound to the sugar at position 5. The term “nucleoside triphosphate” also encompasses nucleoside triphosphate analogues, such as, nucleoside triphosphates with a different sugar and/or a different nitrogenous base than the natural NTPs, as well as nucleoside triphosphates with a modified 2′-OH, 3′-OH and/or 5′-triphosphate position. In particular, nucleoside triphosphate analogues include those useful for the synthesis of xeno nucleic acids (XNA), as defined hereinabove. Non-limiting examples of such synthetic nucleoside triphosphate analogues are given in FIG. 4 of Chakravarthy et al., 2017 (Theranostics. 7(16):3933-3947), the content of which Figure is herein incorporated by reference. Further non-limiting examples of such synthetic nucleoside triphosphate analogues are given in [0250] to [0280] of US 2009-0286696, the content of which paragraphs is herein incorporated by reference.

A nucleoside triphosphate containing a deoxyribose is typically referred to as deoxynucleoside triphosphate and abbreviated as dNTP. Consistently, a nucleoside triphosphate containing a ribose is typically referred to as ribonucleoside triphosphate and abbreviated as rNTP.

Examples of deoxynucleoside triphosphates include, but are not limited to, deoxyadenosine triphosphate (dATP), deoxyguanosine triphosphate (dGTP), deoxycytidine triphosphate (dCTP), and deoxythymidine triphosphate (dTTP). Further examples of deoxynucleoside triphosphates include deoxyuridine triphosphate (dUTP), deoxyinosine triphosphate (dITP), and deoxyxanthosine triphosphate (dXTP).

Examples of ribonucleoside triphosphates include, but are not limited to, adenosine triphosphate (ATP), guanosine triphosphate (GTP), cytidine triphosphate (CTP) and uridine triphosphate (UTP). Further examples of nucleoside triphosphates include N6-methyladenosine triphosphate (m6ATP), 5-methyluridine triphosphate (m5UTP), 5-methylcytidine triphosphate (m5CTP), pseudouridine triphosphate (WUTP), inosine triphosphate (ITP), xanthosine triphosphate (XTP), and wybutosine triphosphate (yWTP).

Other types of nucleosides may be bound to three phosphates to form nucleoside triphosphates, such as naturally occurring modified nucleosides and artificial nucleosides.

By “selected” with reference to nucleoside triphosphates, it is meant a nucleoside triphosphate or a combination of nucleoside triphosphates purposely chosen among the various possibilities of nucleoside triphosphates, including, but not limited to those described above, with the idea of synthetizing either (1) a nucleic acid with a random sequence or (2) a nucleic acid with a defined nucleotide sequence.

By “combination of nucleoside triphosphates”, it is meant a mix of at least two different nucleoside triphosphates.

In one embodiment, the method of the present invention is a method for template-independent synthesis of nucleic acids with random nucleotide sequence, which comprises—optionally iteratively—contacting an initiator sequence comprising a 3′-end nucleotide with a free 3′-hydroxyl group, with a (optionally, selected) combination of nucleoside triphosphates in the presence of a mutated, functionally active primase domain described above, thereby covalently and randomly binding said combination of (optionally, selected) nucleoside triphosphates to the free 3′-hydroxyl group of the 3′-end nucleotide.

In this embodiment, the (optionally, selected) combination of nucleoside triphosphates do not comprise terminating nucleoside triphosphates.

In one embodiment, the method of the present invention is a method for template-independent, sequence-controlled synthesis of nucleic acids, which comprises iteratively contacting an initiator sequence comprising a 3′-end nucleotide with a free 3′-hydroxyl group of a nucleotide with a selected terminating nucleoside triphosphate in the presence of a mutated, functionally active primase domain described above, thereby covalently binding said selected terminating nucleoside triphosphate to the free 3′-hydroxyl group of the 3′-end nucleotide.

In the latter embodiment of sequence-controlled synthesis of nucleic acids, the 3′-hydroxyl group of a 3′-end nucleotide is contacted with a selected terminating nucleoside triphosphate.

By “terminating nucleoside triphosphate”, also sometimes termed “3′-blocked nucleoside triphosphates” or “3′-protected nucleoside triphosphates”, it is referred to nucleoside triphosphates which have an additional group (hereafter, “3′-blocking group” or “3′-protecting group”) on their 3′-end (i.e., at position 3 of their 5-carbon sugar), for the purpose of preventing further, undesired, addition of nucleoside triphosphates after specific addition of the selected nucleotide (n+1) to a strand of nucleic acid (n).

In one embodiment, the 3′-blocking group may be reversible (can be removed from the nucleoside triphosphate) or irreversible (cannot be removed from the nucleoside triphosphate), i.e., the terminating nucleoside triphosphate may be a reversible terminating nucleoside triphosphate or a non-reversible terminating nucleoside triphosphate.

In one embodiment, the 3′-blocking group is reversible, and removal of the 3′-blocking group from the nucleoside triphosphate (e.g., using a cleaving agent) allows the addition of further nucleoside triphosphate to the synthetized nucleic acid.

Examples of reversible 3′-blocking groups include, but are not limited to, methyl, methoxy, oxime, 2-nitrobenzyl, 2-cyanoethyl, allyl, amine, aminoxy, azidomethyl, tert-butoxy ethoxy (TBE), propargyl, acetyl, quinone, coumarin, aminophenol derivative, ketal, N-methyl-anthraniloyl, and the like.

In the context of the present invention, the term “cleaving agent” refers to any chemical, biological or physical agent which is able to remove (or cleave) a reversible 3′-blocking group from a reversible terminating nucleoside triphosphate.

In one embodiment, the cleaving agent is a chemical cleaving agent. In one embodiment, the cleaving agent is an enzymatic cleaving agent. In one embodiment, the cleaving agent is a physical cleaving agent.

It will be understood by the one skilled in the art that the selection of a cleaving agent is dependent on the type of 3′-blocking group used. For instance, tris(2-carboxyethyl)phosphine (TCEP) can be used to cleave a 3′-O-azidomethyl group, palladium complexes can be used to cleave a 3′-O-allyl group, sodium nitrite can be used to cleave a 3′-aminoxy group, and UV light can be used to cleave a 3′-O-nitrobenzyl group.

In one embodiment, the cleaving agent may be used in conjunction with a cleavage solution comprising a denaturant (such as, e.g., urea, guanidinium chloride, formamide or betaine). In particular, adding a denaturant provides the advantage of disrupting any undesirable secondary structures in the synthetized nucleic acid. The cleavage solution may further comprise one or more buffers, which will be dependent on the exact cleavage chemistry and cleaving agent used.

In one embodiment, the 3′-blocking group is irreversible, and addition of a non-reversible terminating nucleoside triphosphate to the synthetized nucleic acid terminates the synthesis. Such irreversible 3′-blocking groups may be useful, e.g., as fluorophores, labels, tags, etc.

Example of irreversible 3′-blocking groups include, but are not limited to, fluorophores, such as, e.g., methoxycoumarin, dansyl, pyrene, Alexa Fluor 350, AMCA, Marina Blue dye, dapoxyl dye, dialkylaminocoumarin, bimane, hydroxycoumarin, Cascade Blue dye, Pacific Orange dye, Alexa Fluor 405, Cascade Yellow dye, Pacific Blue dye, PyMPO, Alexa Fluor 430, NBD, QSY 35, fluorescein, Alexa Fluor 488, Oregon Green 488, BODIPY 493/503, rhodamine green dye, BODIPY FL, 2′,7′-dichlorofluorescein, Oregon Green 514, Alexa Fluor 514, 4′,5′-dichloro-2′,7′-dimethoxyfluorescein (JOE), eosin, rhodamine 6G, BODIPY R6G, Alexa Fluor 532, BODIPY 530/550, BODIPY TMR, Alexa Fluor 555, tetramethylrhodamine (TMR), Alexa Fluor 546, BODIPY 558/568, QSY 7, QSY 9, BODIPY 564/570, lissamine rhodamine B, rhodamine red dye, BODIPY 576/589, Alexa Fluor 568, X-rhodamine, BODIPY 581/591, BODIPY TR, Alexa Fluor 594, Texas Red dye, naphthofluorescein, Alexa Fluor 610, BODIPY 630/650, malachite green, Alexa Fluor 633, Alexa Fluor 635, BODIPY 650/665, Alexa Fluor 647, QSY 21, Alexa Fluor 660, Alexa Fluor 680, Alexa Fluor 700, Alexa Fluor 750, Alexa Fluor 790, and the like.

Further examples of irreversible 3′-blocking groups include, but are not limited to, biotin or desthiobiotin groups.

In any one of the above embodiments, the nucleoside triphosphate is a 2′-protected nucleoside triphosphate.

By “2′-protected nucleoside triphosphate”, it is referred to nucleoside triphosphates which have an additional group (hereafter, “2′-protecting group”) on their 2′-end (i.e., at position 2 of their 5-carbon sugar). A particular—although not the sole-purpose of such 2′-protecting groups is to protect the reactive 2′-hydroxyl group in the specific case of ribonucleotide triphosphates.

Any 3′-blocking groups described above, whether reversible or irreversible, are also suitable to serve as 2′-protecting groups.

Additionally, any 3′-blocking groups described above, whether reversible or irreversible, can further be added at any position of the nucleoside triphosphates, whether on their 5-carbon sugar moiety and/or on their nitrogenous base.

In one embodiment, the method for template-independent synthesis of nucleic acids comprises the following steps:
a) providing an initiator sequence comprising a 3′-end nucleotide with a free 3′-hydroxyl group;
b) contacting said 3′-end nucleotide with a (optionally, selected) nucleoside triphosphate (or a combination of (optionally, selected) nucleoside triphosphates) in the presence of a mutated, functionally active primase domain described above;
   thereby covalently binding said (optionally, selected) nucleoside triphosphate to the free 3′-hydroxyl group of the 3′-end nucleotide.

In one embodiment, the method according to the present invention is for template-independent synthesis of nucleic acids with a random sequence, and it comprises the following steps:
a) providing an initiator sequence comprising a 3′-end nucleotide with a free 3′-hydroxyl group;
b) contacting said 3′-end nucleotide with a combination of (optionally, selected) nucleoside triphosphates in the presence of a mutated, functionally active primase domain described above;
   thereby randomly covalently binding said combination of (optionally, selected) nucleoside triphosphates to the free 3′-hydroxyl group of the 3′-end nucleotide.

In one embodiment, the method according to the present invention is for template-independent, sequence-controlled synthesis of nucleic acids, and it comprises the following steps:
a) providing an initiator sequence comprising a 3′-end nucleotide with a free 3′-hydroxyl group;
b) contacting said 3′-end nucleotide with a selected reversibly terminating nucleoside triphosphate in the presence of a mutated, functionally active primase domain described above,
   thereby covalently binding said selected reversibly terminating nucleoside triphosphate to the free 3′-hydroxyl group of the 3′-end nucleotide;
c) applying a washing solution to remove all reagents, in particular to remove unbound reversibly terminating nucleoside triphosphates;
d) cleaving the reversible 3′-blocking group of the covalently bound terminating nucleoside triphosphate in the presence of a cleaving agent;
   thereby obtaining a nucleotide with a free 3′-hydroxyl group;
e) optionally, applying a washing solution to remove all reagents, in particular to remove the cleaving agent;
f) optionally, reiterating steps b) to e) multiple times to synthetize a nucleic acid of desired length and nucleotide sequence.

In one embodiment, more than 1 nucleoside triphosphate is added to the 3′-end nucleotide with a free 3′-hydroxyl group, such as, more than 2, 5, 10, 20, 30, 40, 50, 60, 70, 80, 90, 100, 200, 300, 400, 500, 600, 700, 800, 900, 1000, 1250, 1500, 1750, 2000, 2250, 2500, 2750, 3000, 4000, 5000, 6000, 7000, 8000, 9000, 10000 or even more nucleoside triphosphates are added to the 3′-end nucleotide with a free 3′-hydroxyl group by reiterating steps b) to e) as many times.

In one embodiment, the method for template-independent synthesis of nucleic acids according to the present invention is carried out in the presence of one or more buffers (e.g., Tris or cacodylate) and/or one or more salts (e.g., Na⁺, K⁺, Mg²⁺, Mn²⁺, Cu²⁺, Zn²⁺, Co²⁺, etc., all with appropriate counterions, such as Cl⁻).

In one embodiment, the method for template-independent synthesis of nucleic acids according to the present invention is carried out in the presence of one or more divalent cations (e.g., Mg²⁺, Mn²⁺, Co²⁺, etc., all with appropriate counterions, such as Cl⁻), preferably in the presence of Mn²⁺.

In one embodiment, the method for template-independent synthesis of nucleic acids according to the present invention is carried out at a temperature ranging from about from about 60° C. to about 95° C. In one embodiment, the method for template-independent synthesis of nucleic acids according to the present invention is carried out at a temperature of about 60° C., 65° C., 70° C., 75° C., 80° C., 85° C., 90° C. or 95° C.

In one embodiment, the mutated, functionally active primase domain described above may be used in the method for template-independent synthesis of nucleic acids according to the present invention for producing synthetic homo- and heteropolymers. One skilled in the art is familiar with means and methods for producing synthetic homo- and heteropolymers, described in, e.g., Bollum, 1974 (In Boyer [Ed.], The enzymes [3^{rd} Ed., Vol. 10, pp. 145-171]. New York, NY: Academic Press), the content of which is incorporated herein by reference.

In one embodiment, the mutated, functionally active primase domain described above may be used in the method for template-independent synthesis of nucleic acids according to the present invention for homopolymeric tailing of any type of 3′-OH terminus. One skilled in the art is familiar with means and methods for homopolymeric tailing, described in, e.g., Deng & Wu, 1983 (Methods Enzymol. 100:96-116) and Eschenfeldt et al., 1987 (Methods Enzymol. 152:337-342), the content of which is incorporated herein by reference.

In one embodiment, the mutated, functionally active primase domain described above may be used in the method for template-independent synthesis of nucleic acids according to the present invention for oligonucleotide, DNA, and RNA labeling. One skilled in the art is familiar with means and methods for labelling, described in, e.g., Deng & Wu, 1983 (Methods Enzymol. 100:96-116), Tu & Cohen, 1980 (Gene. 10(2):177-183), Vincent et al., 1982 (Nucleic Acids Res. 10(21):6787-6796), Kumar et al., 1988 (Anal Biochem. 169(2):376-382), Gaastra & Klemm, 1984 (In Walker et al. [Eds.], Nucleic acids [Vol. 2, Methods in molecular biology, pp. 269-271]. Clifton, NJ: Humana Press), Igloi & Schiefermayr, 1993 (Biotechniques. 15(3):486-497) and Winz et al., 2015 (Nucleic Acids Res. 43(17):el 10), the content of which is incorporated herein by reference.

In one embodiment, the mutated, functionally active primase domain described above may be used in the method for template-independent synthesis of nucleic acids according to the present invention for 5′-RACE (Rapid Amplification of cDNA Ends). One skilled in the art is familiar with means and methods for 5′-RACE, described in, e.g., Scotto-Lavino et al., 2006 (Nat Protoc. 1(6):2555-62), the content of which is incorporated herein by reference.

In one embodiment, the mutated, functionally active primase domain described above may be used in the method for template-independent synthesis of nucleic acids according to the present invention for in situ localization of apoptosis, such as TUNEL (terminal deoxynucleotidyl transferase dUTP nick end labeling) assay. One skilled in the art is familiar with means and methods for in situ localization of apoptosis such as TUNEL assay, described in, e.g., Gorczyca et al., 1993 (Cancer Res. 53(8):1945-1951) and Lebon et al., 2015 (Anal Biochem. 480:37-41), the content of which is incorporated herein by reference.

In a third aspect, the present invention relates to a system for template-independent synthesis of nucleic acids, comprising:
an initiator sequence comprising a 3′-end nucleotide with a free 3′-hydroxyl group, optionally wherein said initiator sequence is immobilized onto a support;
nucleoside triphosphates; and
a mutated, functionally active primase domain described above.

In one embodiment, the system is suitable for template-independent synthesis of nucleic acids with a random sequence, and it comprises:
an initiator sequence comprising a 3′-end nucleotide with a free 3′-hydroxyl group, optionally wherein said initiator sequence is immobilized onto a support;
a combination of (optionally, selected) nucleoside triphosphates, wherein said (optionally, selected) nucleoside triphosphates are not terminating nucleoside triphosphates; and
a mutated, functionally active primase domain described above.

In one embodiment, the system is suitable for template-independent, sequence-controlled synthesis of nucleic acids, and it comprises:
an initiator sequence comprising a 3′-end nucleotide with a free 3′-hydroxyl group, optionally wherein said initiator sequence is immobilized onto a support;
reversibly terminating selected nucleoside triphosphates, wherein different nucleoside triphosphates are not combined together in a same vial;
a cleaving agent; and
a mutated, functionally active primase domain described above.

In a fourth aspect, the present invention relates to a kit comprising:
an initiator sequence comprising a 3′-end nucleotide with a free 3′-hydroxyl group, optionally wherein said initiator sequence is immobilized onto a support;
nucleoside triphosphates; and
a mutated, functionally active primase domain described above.

In one embodiment, the kit comprises:
an initiator sequence comprising a 3′-end nucleotide with a free 3′-hydroxyl group, optionally wherein said initiator sequence is immobilized onto a support;
a combination of (optionally, selected) nucleoside triphosphates, wherein said (optionally, selected) nucleoside triphosphates are not terminating nucleoside triphosphates; and
a mutated, functionally active primase domain described above.

In one embodiment, the kit comprises:
an initiator sequence comprising a 3′-end nucleotide with a free 3′-hydroxyl group, optionally wherein said initiator sequence is immobilized onto a support;
reversibly terminating selected nucleoside triphosphates, wherein different nucleoside triphosphates are not combined together in a same vial;
a cleaving agent; and
a mutated, functionally active primase domain described above.

### BRIEF DESCRIPTION OF THE DRAWINGS

FIG. 1 is a bar chart representing the template-independent nucleic acid synthesis relative activity of the purified mutants relative to the activity of PolpP12_{Δ297-898} (SHEE-WT), set at 100%. Reactions were performed at 70° C. using a dNTP mix and a 20-nucleotide primer as substrates. Activity represents the length of the elongated DNA.

FIG. 2 is a bar chart representing the template-independent nucleic acid synthesis relative activity of double mutants, relative to the activity of PolpP12_{Δ297-898} (SHEE-WT), set at 100%. Reactions were performed at 70° C. using a dNTP mix and a 20-nucleotide primer as substrates. Activity represents the length of the elongated DNA.

FIG. 3 is a photograph of a 1.5% agarose gel electrophoresis showing a template independent nucleic acid synthesis assay using PolpP12_{Δ297-898} (SHEE-WT), in comparison with triple mutants. Reactions were performed at 70° C. using a dNTP mix and a 20-nucleotides primer as substrates. Ladder: SmartLadder 200 to 10000 bp (Eurogentec).

FIG. 4 is a bar chart representing a template independent nucleic acid synthesis activity of triple, relative to the activity of PolpP12_{Δ297-898} (SHEE-WT), set at 100%. Activity represents the length of the elongated DNA obtained from the agarose gel depicted in FIG. 4.

FIG. 5 is a bar chart representing a template independent nucleic acid synthesis activity of PolpP12_{Δ297-898} (SHEE-WT) fused to a DNA-binding domain from Thermotoga neapolitana either at the N-terminus (SHEE-N18) or the C-terminus (SHEE-C18), relative to the activity of PolpP12Δ297-898 (SHEE-WT), set at 100%. Activity represents the length of the elongated DNA.

FIG. 6 is a photograph of an electrophoresis gel (SDS-PAGE) showing the purification of respectively PolpTN2Δ311-923, PolpTN2Δ311-923_B, PolpTN2Δ311-923_C and PolpTN2Δ311-923_D. MW ladder: molecular weight ladder (left lane).

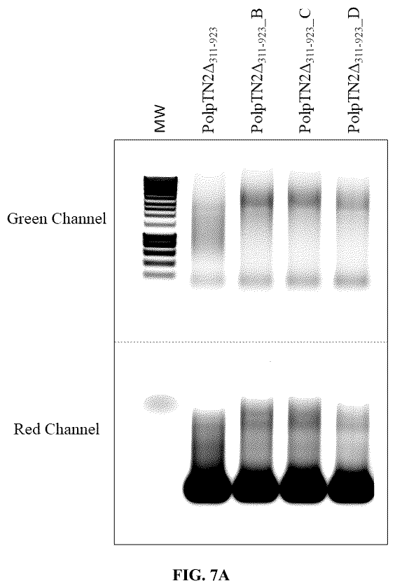

FIGS. 7A-B are a set of two photographs of a 1.5% agarose gel electrophoresis showing a template-independent nucleic acid synthesis assay or a template-independent ab-inito synthesis assay using PolpTN2Δ₃₁₁₋₉₂₃, PolpTN2Δ_{311-923_B}, PolpTN2Δ₃₁₁₋₉₂₃_C or PolpTN2Δ₃₁₁₋₉₂₃_D and carried out in the presence or in the absence of dNTPs and/or the initiator sequence (bearing the Cy5 fluorophore in 5′). Reactions were performed at 70° C. for 10 min in the presence or in the absence of each substrate. MW ladder is SmartLadder 200 to 10000 bp (Eurogentec). Green channel, MidoriGreen direct fluorescence at 520 nm. Red channel, Cy5 fluorescence at 675 nm. FIG. 7A: in the presence of dNTPs and the initiator sequence (bearing the Cy5 fluorophore in 5′); FIG. 7B: in the presence of dNTPs and in the absence of the initiator sequence.

### EXAMPLES

The present invention is further illustrated by the following examples.

The Inventors have previously identified several archaeal DNA primase domains that are capable of template-independent synthesis, at temperatures between 60° C. and 95° C. (International patent applications WO 2021/250269 and WO 2021/250265).

In brief, the data surprisingly showed that:
PolpP12_{Δ297-898} has template-independent terminal nucleotidyl transferase activity and is devoid of ab-initio single-stranded nucleic acid synthesis activity (Example 2 of WO 2021/250269);
PolpP12_{Δ297-898} has a higher processivity than a member of X-family polymerases, the recombinant terminal deoxynucleotidyl transferase TdT (Example 3 of WO 2021/250269);
PolpP12_{Δ297-898} is capable of incorporating protected deoxyribonucleosides triphosphate and protected ribonucleosides triphosphate (Examples 4 and 6 of WO 2021/250269);
PolpP12_{Δ297-898} is capable of incorporating ribonucleosides triphosphate and deoxyuridine triphosphate (Example 5 of WO 2021/250269);
PolpP12_{Δ297-898} is capable of incorporating deoxyinosine triphosphate and base-modified nucleosides triphosphate (Example 7 of WO 2021/250269);
PolpP12_{Δ297-898} or PolpTN2_{Δ311-923} variants with internal loop deletions are still functional (Example 8 of WO 2021/250269; Example 2 of WO 2021/250265); and
PolpTN2_{Δ311-923}, PolpTCIR10_{Δ303-928}, PolpTpep_{Δ295-914} and PolpTcele_{Δ295-913} have ab-initio single-stranded nucleic acid synthesis activity (Examples 1 and 3-4 of WO 2021/250265).

Here, the Inventors aimed at providing mutants of these archaeal DNA primase domains with at least equivalent if not improved template-independent terminal nucleotidyl transferase activity compared to their wild-type counterparts.

### Example 1

### Single Point Mutations of PolpP12_{Δ297-898} Modulate the Template-Independent Terminal Nucleotidyl Transferase Activity

Around 90 single point mutants of the N-terminal domain of the DNA primase from Pyrococcus sp. 12-1 (PolpP12_{Δ297-898} having the amino acid sequence of SEQ ID NO: 2) were generated using the Q5® Site-Directed Mutagenesis kit (New England Biolabs) following manufacturer's protocol. After sequence verification using Sanger's sequencing, PolpP12_{Δ297-898} (SHEE-WT) and its corresponding mutants were expressed and purified following a protocol adapted from International patent publication WO2011098588 and Gill et al., 2014 (Nucleic Acids Res. 42(6):3707-3719).

To evaluate the gain or the loss of activity of PolpP12_{Δ297-898} mutants, a template-independent nucleic acid synthesis assay was carried out using PolpP12_{Δ297-898} (SHEE-WT) as positive control. All experiments were run at 70° C. using a dNTP mix as substrate and a single stranded nucleic acid primer as initiator sequence. After reaction, samples were resolved on 1.5% agarose gels and nucleic acids were stained using Midori Green Direct. For each mutant, the length of the synthetized DNA was determined using a calibration curve made from the DNA ladder (SmartLadder 200 to 10000 bp, Eurogentec) and relative activities were calculated by setting the activity of PolpP12_{Δ297-898} (SHEE-WT) as 100%.

TABLE 1
single point mutations of PolpP12_{Δ297-898.} Amino acid
residue positions with reference to SEQ ID NO: 2 numbering.

| Mutant ID | Substitution |
|---|---|
| SHEE-3.12 | F219Y |
| SHEE-3.21 | Y233A |
| SHEE-3.24 | Y233H |
| SHEE-3.32 | Y235F |
| SHEE-3.33 | Y235W |
| SHEE-4.2 | N217R |
| SHEE-4.3 | N217K |
| SHEE-9.1 | Y122A |
| SHEE-9.2 | Y122H |
| SHEE-10.1 | F74Y |
| SHEE-11.1 | K234R |
| SHEE-12.1 | K236R |
| SHEE-13.1 | N206R |
| SHEE-15.1 | T230C |
| SHEE-15.2 | T230A |
| SHEE-17.1 | L229A |
| SHEE-18.1 | I231A |
| SHEE-20.3 | S68N |
| SHEE-21.3 | F74Q |
| SHEE-23.2 | F174R |
| SHEE-26.2 | P228N |
| SHEE-26.3 | P228A |
| SHEE-27.1 | L229G |
| SHEE-27.2 | L229N |
| SHEE-27.3 | L229R |
| SHEE-28.1 | T230S |
| SHEE-29.2 | I231R |
| SHEE-29.3 | I231K |
| SHEE-30.1 | N232R |
| SHEE-31.1 | Y233R |
| SHEE-31.2 | Y233K |
| SHEE-33.1 | P238R |

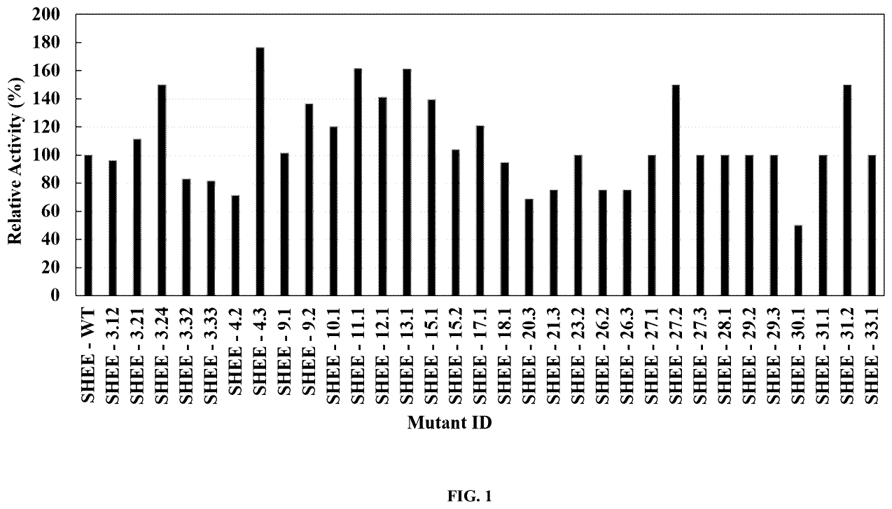

FIG. 1 shows the template-independent nucleic acid synthesis relative activity of the purified mutants of Table 1, relative to the activity of PolpP12_{Δ297-898} (SHEE-WT).

Strikingly, SHEE-3.24, SHEE-4.3, SHEE-9.2, SHEE-10.1, SHEE-11.1, SHEE-12.1, SHEE-13.1, SHEE-15.1, SHEE-17.1, SHEE-27.2 and SHEE-31.2 showed a clear increase of activity ranging from 120% to 180% compared to wild-type PolpP12_{Δ297-898} (SHEE-WT).

Surprising, SHEE-4.3, SHEE-10.1 and SHEE-13.1 showed an improved activity, although their mutated amino acid positions (N217K, F74Y and N206R, respectively) are expected to be located outside the catalytic pocket. This phenomenon suggests that these residues are involved in an improved binding of the initiator DNA primer, thus leading to an enhanced template-independent nucleic acid synthesis activity.

### Example 2

Multiple Point Mutations of PolpP12_{Δ297-898} have an Additive Effect on the Improvement of the Template-Independent Terminal Nucleotidyl Transferase Activity

Multiple point mutations of the N-terminal domain of the DNA primase from Pyrococcus sp. 12-1 (PolpP12_{Δ297-898} having the amino acid sequence of SEQ ID NO: 2) were generated using the Q5® Site-Directed Mutagenesis kit (New England Biolabs) following manufacturer's protocol. Mutants were designed based on the results of single point mutations described in Example 1. After sequence verification using Sanger's sequencing, PolpP12_{Δ297-898} (SHEE-WT) and its corresponding mutants were expressed and purified following a protocol adapted from International patent publication WO2011098588 and Gill et al., 2014 (Nucleic Acids Res. 42(6):3707-3719) (Table 2).

TABLE 2
multiple point mutations of PolpP12_{Δ297-898.} Amino acid
residue positions with reference to SEQ ID NO: 1 numbering.

| Mutant ID | Substitution |
|---|---|
| SHEE-4.3/11.1 | N217K/K234R |
| SHEE-4.3/12.1 | N217K/K236R |
| SHEE-4.3/13.1 | N217K/N206R |
| SHEE-9.2/3.24 | Y122H/Y233H |
| SHEE-9.2/4.3 | Y122H/N217K |
| SHEE-9.2/11.1 | Y122H/K234R |
| SHEE-9.2/12.1 | Y122H/K236R |
| SHEE-9.2/13.1 | Y122H/N206R |
| SHEE-9.2/15.1 | Y122H/T230C |
| SHEE-10.1/4.3 | F74Y/N217K |
| SHEE-10.1/11.1 | F74Y/K234R |
| SHEE-11.1/15.1 | K234R/T230C |
| SHEE-12.1/15.1 | K236R/T230C |
| SHEE-13.1/3.24 | N206R/Y233H |
| SHEE-13.1/15.1 | N206R/T230C |
| SHEE-15.1/4.3 | T230C/N217K |
| SHEE-4.3/13.1/3.24 | N217K/N206R/Y233H |
| SHEE-4.3/13.1/9.2 | N217K/N206R/Y122H |
| SHEE-4.3/13.1/11.1 | N217K/N206R/K234R |

To evaluate the gain or the loss of activity of the multiple point mutants of PolpP12_{Δ297-898}, a template-independent nucleic acid synthesis assay was carried out using PolpP12_{Δ297-898} (SHEE-WT) as positive control. All experiments were run at 70° C. using a dNTP mix as substrate and a single stranded nucleic acid primer as initiator sequence. After reaction, samples were resolved on 1.5% agarose gels and nucleic acids were stained using Midori Green Direct. For each mutant, the length of the synthetized DNA was determined using a calibration curve made from the DNA ladder (SmartLadder 200 to 10000 bp, Eurogentec) and relative activities were calculated by setting the activity of PolpP12_{Δ297-898} (SHEE-WT) as 100%.

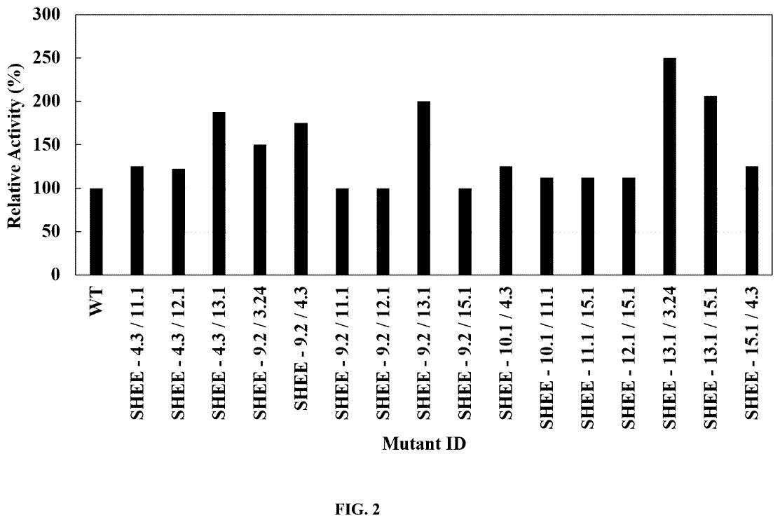

As shown in FIG. 2, all tested double mutants display at least an equivalent activity to the wild-type PolpP12_{Δ297-898} (SHEE-WT).

Interestingly, several double mutants even showed an increase of activity when compared to single mutants. Most striking, major improvements are obtained with the 13.1/3.24, 13.1/15.1, 9.2/13.1, 4.3/13.1 and 9.2/4.3, combinations.

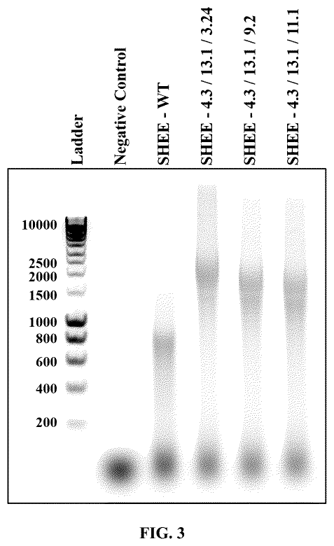

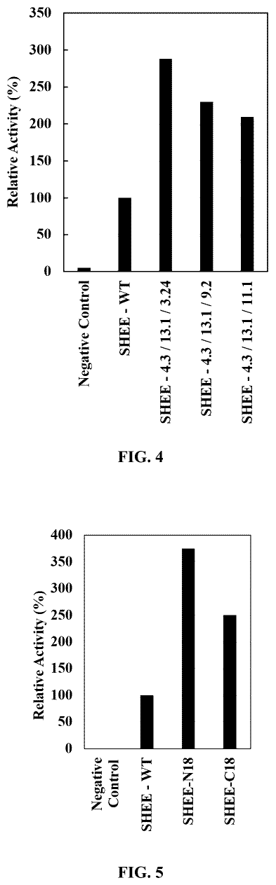

Likewise, as shown in FIGS. 3 and 4, the three tested triple mutants showed an increased activity ranging from 200% to 300% compared to wild-type PolpP12_{Δ297-898} (SHEE-WT).

Surprisingly, the activity of these multiple point mutants of PolpP12_{Δ297-898} were much higher than the ones obtained for their corresponding single mutants (Example 1), suggesting an additive effect of the mutated residues.

Of note, two out of three mutated positions in the triple mutants are expected to be located outside the catalytic pocket (N217K and N206R). This phenomenon thus reinforces the hypothesis of an improved binding of the initiator DNA primer, which leads to an enhanced template-independent nucleic acid synthesis activity.

### Example 3

Addition of a ssDNA Binding Domain Improves the Template-Independent Terminal Nucleotidyl Transferase Activity

In addition to mutagenesis studies, we sought to study the fusion of PolpP12_{Δ297-898} (SHEE-WT) with processivity factors to improve template-independent terminal nucleotidyl transferase activity.

For that purpose, two constructs were prepared using a DNA-binding domain from Thermotoga neapolitana (with SEQ ID NO: 27), fused either at the N-terminus (SHEE-N18, with SEQ ID NO: 28) or the C-terminus (SHEE-C18, with SEQ ID NO: 29) of the wild-type PolpP12_{Δ297 898}.

SEQ ID NO: 28-SHEE-N18
MSFFNRIILIGRLVRDPEERYTLSGTPVTTFTIAVDRVPRKNAPDDAQTTDFFRV
VTFGRLAEFARTYLTKGRLILVEGEMRMRRWETQTGEKRVSPEVVANVVRFM
DRKPVEMPSEDIEEKLEIPEEDFTDDTFSEDEPPFRWGSELEMRPSDIIIDVYKAIQ
DHPGAGKLAIELRFYPRPTSEWIIVADIEDKAEELHKVLFKNNVLGKKEAYISM
ALHDFEEVGKKLEKLRELEEERAQKEGRKPREVTLRNVQGEATGKVHKTVSK
YTLTLVVDIDVEEIHKSKVVESEEKAFELAKRAWDELKPKLEGIGVKPRYVFFT
GGGVQLWFVAPGLEPIEVIDRASRVIPPVLNAMLPEGYSVDNIFDRARIVRVPLT
INYKYKTPDERPLEIRGRLIEFNDVRTPLGEVLDKLEAYAKEHGISLVTPSQARFI
GTVGRYEVDKGTGT
SEQ ID NO: 29-SHEE-C18
MRPSDIIIDVYKAIQDHPGAGKLAIELRFYPRPTSEWIIVADIEDKAEELHKVLFK
NNVLGKKEAYISMALHDFEEVGKKLEKLRELEEERAQKEGRKPREVTLRNVQG
EATGKVHKTVSKYTLTLVVDIDVEEIHKSKVVESEEKAFELAKRAWDELKPKL
EGIGVKPRYVFFTGGGVQLWFVAPGLEPIEVIDRASRVIPPVLNAMLPEGYSVD
NIFDRARIVRVPLTINYKYKTPDERPLEIRGRLIEFNDVRTPLGEVLDKLEAYAK
EHGISLVTPSQARFIGTVGRYEVDKGTMEFEASFFNRIILIGRLVRDPEERYTLSG
TPVTTFTIAVDRVPRKNAPDDAQTTDFFRVVTFGRLAEFARTYLTKGRLILVEG
EMRMRRWETQTGEKRVSPEVVANVVRFMDRKPVEMPSEDIEEKLEIPEEDFTD
DTFSEDEPPF

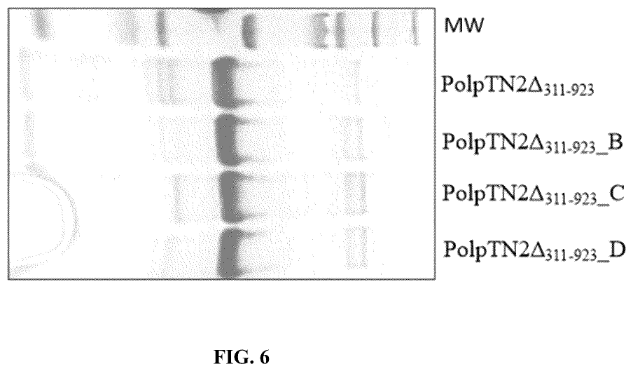

As shown in FIG. 5, both fusion variants of PolpP12_{Δ297-898} demonstrate an increased activity, ranging from 250% to more than 350%, for SHEE-C18 and SHEE-N18 respectively, indicating that increasing the binding of DNA drastically influences the template-independent terminal nucleotidyl transferase activity.

Thus, these variants might be associated to single point mutations or a combination of mutations, such as the ones described in Examples 1 and 2, to further increase even more their terminal nucleotidyl transferase activity.

### Example 4

Multiple Point Mutations of PolpP12_{Δ297-898} are Transposable to _{PolpTN2Δ311-923} for the Improvement of the Template-Independent Terminal Nucleotidyl Transferase Activity

To further investigate the potency of multiple point mutations of the N-terminal domain of the DNA primase from Pyrococcus sp. 12-1 described in Example 2 (PolpP12_{Δ297-898} having the amino acid sequence of SEQ ID NO: 2) most active mutations were subsequently transposed to the N-terminal domain of the DNA primase from Thermococcus nautili sp. 30-1 (PolpTN2x311-923 having the amino acid sequence of SEQ ID NO: 5) generated from gene synthesis service (Twist Bioscience).

N-terminal domain of the DNA primase from Thermococcus nautili sp. 30-1 was considered has a representative member of the Thermococcus genus, including the N-terminal domain of the DNA primase from Thermococcus sp. CIR10, Thermococcus peptonophilus and Thermococcus celericrescens, which were all previously shown to exhibit both template independent nucleotidyl transferase activity and template independent ab-initio activity.

PolpTN2_{Δ311-923} and its corresponding mutants were expressed and purified following a protocol adapted from International patent publication WO2011098588 and Gill et al., 2014 (Nucleic Acids Res. 42(6):3707-3719) (Table 3).

TABLE 3
multiple point mutations of PolpP12_{Δ297-898.} Amino acid
residue positions with reference to SEQ ID NO: 5 numbering.

| Mutant ID | Substitution |
|---|---|
| PolpTN2Δ₃₁₁₋₉₂₃ | WT |
| PolpTN2Δ₃₁₁₋₉₂₃_{_{—}}B | F121H/N205R/N222K |
| PolpTN2Δ₃₁₁₋₉₂₃_{_{—}}C | N205R/N222K/K239R |
| PolpTN2Δ₃₁₁₋₉₂₃_{_{—}}D | F121H/N205R/N222K/K239R |

The amino acid sequence of the wild-type primase domain of the Thermococcus nautili sp. 30-1 DNA primase (herein termed “PolpTN2_{Δ311-923}_B”), with multiple point mutations F121H, N205R and N222K with reference to SEQ ID NO: 5 numbering, is as set forth in SEQ ID NO: 30.

The amino acid sequence of the wild-type primase domain of the Thermococcus nautili sp. 30-1 DNA primase (herein termed “PolpTN2_{Δ311-923}_C”), with multiple point mutations N205R, N222K and K239R with reference to SEQ ID NO: 5 numbering, is as set forth in SEQ ID NO: 31.

The amino acid sequence of the wild-type primase domain of the Thermococcus nautili sp. 30-1 DNA primase (herein termed “PolpTN2_{Δ311-923}_D”), with multiple point mutations F121H, N205R, N222K and K239R with reference to SEQ ID NO: 5 numbering, is as set forth in SEQ ID NO: 32.

To evaluate the gain or the loss of activity of the multiple point mutants of PolpTN2_{Δ311-923}, both a template-independent nucleic acid synthesis assay and an ab-initio synthesis assay were carried out using PolpTN2_{Δ311-923} as positive control. All experiments were run at 70° C. using a dNTP mix as substrate and, in the presence, or in the absence of a single stranded nucleic acid primer as initiator sequence. After a short reaction period, samples were resolved on 1.5% agarose gels and nucleic acids were stained using Midori Green Direct.

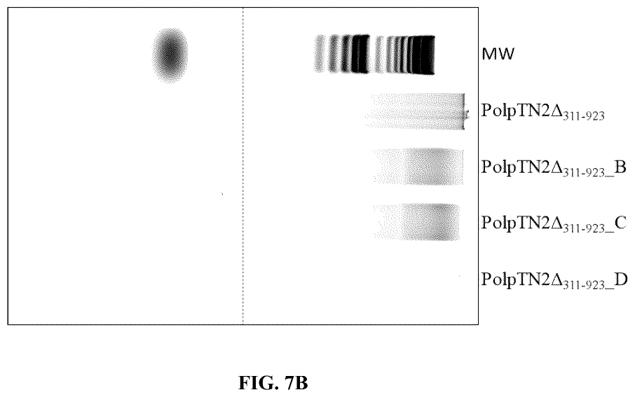

As shown in FIG. 7A, all tested multiple mutants display an increase of template-independent nucleic acid synthesis activity when compared to PolpTN2311-923. This increase of activity is reflected by the higher migration pattern of multiple mutants that can be observed in both green channel (total nucleic acids) and red channel (labeled primer). Such a result thus indicates that the selected mutations of PolpP12_{Δ297-898} can be efficiently transposed to PolpTN2_{Δ311-923}, the representative of the Thermococcus genus.

Surprisingly, the ab-initio activity of these multiple point mutants was lower than PolpTN2_{Δ311-923}, as shown in FIG. 7B, contrasting with the results obtained in the template-independent nucleic acid synthesis experiment (FIG. 7A). Indeed, while the synthesis product of PolpTN2_{Δ311-923} remains at the top of the gel, such a decrease of activity is reflected by the lower migration pattern of multiple mutants that can be observed in green channel (total nucleic acids). Most striking, while PolpTN2_{Δ311-923}_B and PolpTN2_{Δ311-923}_C exhibit a moderate loss of activity, PolpTN2_{Δ311-923}_D was found to be inefficient in performing ab-initio synthesis.

Although this result appears puzzling at a first sight, it actually reinforces the previous hypothesis that N205R and N222K mutations, which correspond to N206R and N217K in PolpTN2311-923, are involved in an improved binding of the initiator DNA primer, thus leading to an enhanced template-independent nucleic acid synthesis activity (FIG. 7A). In contrast, for both F121H and K239R, which are expected to be located within the active site, their mutation might rather play a role in the lower ab-initio activity observed for PolpTN2_{Δ311-923}_B and PolpTN2_{Δ311-923}_C, their association being cumulative as exemplified by PolpTN2_{Δ311-923}_D, which is almost devoid of ab-initio activity (FIG. 7B).

### CONCLUSION

The Inventors have previously discovered that PolpP12_{Δ297-898} (SHEE-WT) exhibit a template-independent nucleic acid synthesis activity in presence of an initiator primer and nucleosides triphosphate—whether unprotected or 3′-O protected, regardless of the size of the protecting group, and whether deoxyribonucleotides or ribonucleotides—, while being devoid of ab-initio nucleic acid synthesis activity.

Here, they provide with mutants of PolpP12297-898 that also exhibit a template-independent nucleic acid synthesis activity. Some of the single-point mutants disclosed herein are shown to exhibit improved template-independent terminal nucleotidyl transferase activity compared to PolpP12_{Δ297-898} (SHEE-WT); while combinations of two or three single-point mutations, and possibly more, have an additive effect on the improvement of the template-independent terminal nucleotidyl transferase activity.

The Inventors had previously discovered that other archaeal DNA primases domains from archaeal DNA primases belonging to the primase-polymerase family also exhibit a template-independent nucleic acid synthesis activity, in particular archaeal DNA primases from Thermococcus nautili sp. 30-1, Thermococcus sp. CIR10, Thermococcus peptonophilus, and Thermococcus celericrescens. Given that the identified positions in PolpP12_{Δ297-898} are conserved in these other DNA primases, as assessed by sequence alignments, it is shown that the mutations described in PolpP12_{Δ297-898} at positionally equivalent positions in these other DNA primases have the same effect.

Finally, the Inventors herein show that fusion of wild-type PolpP12297-898 with oligonucleotide binding domains drastically increase the template-independent terminal nucleotidyl transferase activity. This increase is also expectable with fusions comprising mutants of PolpP12_{Δ297-898} or of other DNA primases.

1. A functionally active mutated primase domain, wherein:
said mutated primase domain comprises the N-terminal domain of a Pyrococcus sp. 12-1 archaeal DNA primase belonging to the primase-polymerase family and shares at least 80% of sequence identity with the primase domain of SEQ ID NO: 2 or SEQ ID NO: 3;
said mutated primase domain comprises at least one amino acid substitution at position N217, K234, N206, L229, Y233, K236, T230, Y122, F74, F174, F219, 1231, P238, Y235, P228, S68, and/or N232 in the amino acid sequence of SEQ ID NO: 2, or at equivalent positions in SEQ ID NO: 3 as determined by sequence and/or structural alignment; and
said mutated primase domain retains at least 50% of the template-independent terminal nucleotidyl transferase activity of the primase domain with SEQ ID NO: 2 or SEQ ID NO: 3.

2. The functionally active mutated primase domain according to claim 1, wherein the at least one amino acid substitution is at position N217, K234, N206, L229, Y233, K236, T230, Y122, F74, F174, F219, 1231, and/or P238 in the amino acid sequence of SEQ ID NO: 2 or at a positionally equivalent position in SEQ ID NO: 3, wherein said mutated primase domain has at least an equivalent template-independent terminal nucleotidyl transferase activity compared to the corresponding wild-type primase domain.

3. The functionally active mutated primase domain according to claim 1, wherein the at least one amino acid substitution is at position N217, K234, N206, L229, Y233, K236, T230, Y122, and/or F74 in the amino acid sequence of SEQ ID NO: 2 or at a positionally equivalent position in SEQ ID NO: 3, wherein said mutated primase domain has an improved template-independent terminal nucleotidyl transferase activity compared to the corresponding wild-type primase domain.

4. The functionally active mutated primase domain according to claim 1, wherein the at least one amino acid substitution comprises:
a) a substitution of N217 with an amino acid residue comprising a positively charged side chain such as a lysine (K), arginine (R), or histidine (H) residue;
b) a substitution of K234 with an amino acid residue comprising a positively charged side chain such as an arginine (R), or histidine (H) residue;
c) a substitution of N206 with an amino acid residue comprising a positively charged side chain such as an arginine (R), histidine (H), or lysine (K) residue;
d) a substitution of L229 with an amino acid residue comprising an amine-containing side chain such as an asparagine (N), arginine (R), glutamine (Q), or lysine (K) residue, or a hydrophobic side chain such as an alanine (A), glycine (G), valine (V), isoleucine (I), or methionine (M) residue;
e) a substitution of Y233 with an amino acid residue comprising a positively charged side chain such as a lysine (K), histidine (H) or arginine (R) residue, or a hydrophobic side chain such as an alanine (A), glycine (G), valine (V), isoleucine (I), leucine (L), or methionine (M) residue;
f) a substitution of K236 with an amino acid residue comprising a positively charged side chain such as an arginine (R), or histidine (H) residue;
g) a substitution of T230 with a cysteine (C) residue, or an amino acid residue comprising a hydrophobic side chain such as an alanine (A), glycine (G), valine (V), isoleucine (I), leucine (L), or methionine (M) residue, or a polar uncharged side chain such as a serine (S), asparagine (N), glutamine (Q), or threonine (T) residue;
h) a substitution of Y122 with an amino acid residue comprising an aromatic side chain such as a histidine (H), phenylalanine (F), or tryptophan (W) residue, or a hydrophobic side chain such as an alanine (A), glycine (G), valine (V), isoleucine (I), leucine (L), or methionine (M) residue;
i) a substitution of F74 with an amino acid residue comprising an aromatic side chain such as a tyrosine (Y), tryptophane (W), or histidine (H) residue, or an amine-containing side chain such as a glutamine (Q), asparagine (N), arginine (R) or lysine (K) residue;
j) a substitution of F174 with an amino acid residue comprising an amine-containing side chain such as an arginine (R), glutamine (Q), asparagine (N), or lysine (K) residue, or an amino acid residue comprising an aromatic side chain such as a tyrosine (Y), tryptophane (W), or histidine (H) residue;
k) a substitution of F219 with an amino acid residue comprising an aromatic side chain such as a tyrosine (Y), histidine (H), or tryptophan (W) residue;
l) a substitution of 1231 with an amino acid residue comprising a hydrophobic side chain such as an alanine (A), glycine (G), valine (V), leucine (L), or methionine (M) residue, or a positively charged side chain such as a lysine (K), arginine (R), or histidine (H) residue;
m) a substitution of P238 with an amino acid residue comprising a positively charged side chain such as an arginine (R), histidine (H), or lysine (K) residue;
n) a substitution of Y235 with an amino acid residue comprising an aromatic side chain such as a phenylalanine (F), tryptophan (W), or histidine (H) residue;
o) a substitution of P228 with an amino acid residue comprising a hydrophobic side chain such as an alanine (A), glycine (G), valine (V), isoleucine (I), leucine (L), or methionine (M) residue, or a polar uncharged side chain such as an asparagine (N), glutamine (Q), threonine (T) or serine (S) residue;
p) a substitution of S68 with an amino acid residue comprising a polar uncharged side chain such as an asparagine (N), glutamine (Q), or threonine (T) residue; or
q) a substitution of N232 with an amino acid residue comprising an amine-containing side chain such as an arginine (R), asparagine (N), glutamine (Q), lysine (K) or histidine (H) residue;
in the amino acid sequence of SEQ ID NO: 2 or at a positionally equivalent position in SEQ ID NO: 3.

5. The functionally active mutated primase domain according to claim1, wherein the at least one amino acid substitution comprises:
a) a substitution of N217 with a lysine (K), or arginine (R) residue; preferably with a lysine (K) residue;
b) a substitution of K234 with an arginine (R) residue;
c) a substitution of N206 with an arginine (R) residue;
d) a substitution of L229 with an asparagine (N), alanine (A), glycine (G), or arginine (R) residue; preferably with an asparagine (N) or alanine (A) residue;
e) a substitution of Y233 with a lysine (K), histidine (H), arginine (R), or alanine (A) residue; preferably with a lysine (K) or histidine (H) residue;
f) a substitution of K236 with an arginine (R) residue;
g) a substitution of T230 with a cysteine (C), alanine (A) or serine (S) residue;
preferably with a cysteine (C) residue;
h) a substitution of Y122 with an alanine (A), or histidine (H) residue;
preferably with a histidine (H) residue;
i) a substitution of F74 with a tyrosine (Y), or glutamine (Q) residue; preferably with a tyrosine (Y) residue;
j) a substitution of F174 with an arginine (R) residue;
k) a substitution of F219 with a tyrosine (Y) residue;
l) a substitution of 1231 with an alanine (A), lysine (K) or arginine (R) residue;
m) a substitution of P238 with an arginine (R) residue;
n) a substitution of Y235 with a phenylalanine (F) or tryptophan (W) residue;
preferably with a phenylalanine (F) residue;
o) a substitution of P228 with an alanine (A) or asparagine (N) residue;
p) a substitution of S68 with an asparagine (N) residue; or
q) a substitution of N232 with an arginine (R) residue;
in the amino acid sequence of SEQ ID NO: 2 or at a positionally equivalent position in SEQ ID NO: 3.

6. The functionally active mutated primase domain according to claim 1, wherein the at least one amino acid substitution is N217K, N206R, K234R, L229N, Y233H, Y233K, K236R, T230C, Y122H, F74Y, L229A, F174R, Y122A, F219Y, L229G, L229R, T230A, T230S, 1231A, 1231R, 1231K, Y233A, Y233R, P238R, Y235F, Y235W, F74Q, P228N, P228A, N217R, S68N, and/or N232R in the amino acid sequence of SEQ ID NO: 2 or at a positionally equivalent position in SEQ ID NO: 3.

7. The functionally active mutated primase domain according to claim 1, comprising at least two amino acid substitutions at positions N217 and K234; N217 and K236; N217 and N206; Y122 and Y233; Y122 and N217; Y122 and K234; Y122 and K236; Y122 and N206; Y122 and T230; F74 and N217; F74 and K234; K234 and T230; K236 and T230; N206 and Y233; N206 and T230; or T230 and N217; in the amino acid sequence of SEQ ID NO: 2 or at a positionally equivalent position in SEQ ID NO: 3.

8. The functionally active mutated primase domain according to claim 1, comprising at least three amino acid substitutions at positions positionally equivalent to N217, N206 and Y233; N217, N206 and Y122; or N217, N206 and K234; in the amino acid sequence of SEQ ID NO: 2 or at a positionally equivalent position in SEQ ID NO: 3.

9. The functionally active mutated primase domain according to claim 1, wherein said mutated primase domain is devoid of ab-initio single-stranded nucleic acid synthesis activity.

10. The functionally active mutated primase domain according to claim 1, wherein the primase domain is fused in N-terminal or C-terminal to a processivity factor through a linker, wherein the processivity factor is a single-stranded DNA-binding protein.

11. The functionally active mutated primase domain according to claim 10, wherein the single-stranded DNA-binding protein is from Thermotoga neapolitana.

12. A nucleic acid encoding the functionally active mutated primase domain according to claim 1, an expression vector comprising said nucleic acid, or a host cell comprising said expression vector.

13. A method for template-independent synthesis of nucleic acids, comprising iteratively contacting an initiator sequence comprising a 3′-end nucleotide with a free 3′-hydroxyl group, with at least one nucleoside triphosphate, or a combination of nucleoside triphosphates, in the presence of a functionally active mutated primase domain according to claim 1, thereby covalently binding said nucleoside triphosphate to the free 3′-hydroxyl group of the 3′-end nucleotide.

14. The method according to claim 13, wherein the template-independent synthesis of nucleic acids is carried out at a temperature ranging from about 60° C. to about 95° C.

15. The method according to claim 13, wherein the initiator sequence is a single stranded nucleic acid primerimmobilized onto a support.

16. The method according to claim 15, wherein said method is:
for template-independent synthesis of nucleic acids with random nucleotide sequence, and the at least one nucleoside triphosphate, or the combination of nucleoside triphosphates, does not comprise terminating nucleoside triphosphates; or
for template-independent sequence-controlled synthesis of nucleic acids, and the at least one nucleoside triphosphate is a terminating nucleoside triphosphate comprising a reversible 3′-blocking group.

17. The method according to claim 13, comprising the steps of:
a) providing the initiator sequence comprising a 3′-end nucleotide with a free 3′-hydroxyl group;
b) contacting said 3′-end nucleotide with a reversibly terminating nucleoside triphosphate in the presence of the functionally active mutated primase domain according to claim 1,
thereby covalently binding said reversibly terminating nucleoside triphosphate to the free 3′-hydroxyl group of the 3′-end nucleotide;
c) applying a washing solution to remove all reagents, in particular to remove unbound reversibly terminating nucleoside triphosphates;
d) cleaving the reversible 3′-blocking group of the covalently bound terminating nucleoside triphosphate in the presence of a cleaving agent;
thereby obtaining a nucleotide with a free 3′-hydroxyl group.

18. The method according to claim 13, further comprising applying a washing solution to remove all reagents, in particular to remove the cleaving agent.

19. The method according to claim 17, further comprising reiterating steps b) to e) multiple times to synthetize the nucleic acid until desired length and nucleotide sequence is achieved.

20. A kit comprising:
a) an initiator sequence comprising a 3′-end nucleotide with a free 3′-hydroxyl group, optionally wherein said initiator sequence is immobilized onto a support;
b) nucleoside triphosphates; and
c) a functionally active mutated primase domain according to claim 1.


SEQUENCE LISTING (32 sequences)

SEQ ID NO: 1 - protein, 898 aa, Pyrococcus sp.
  REGION 1..898: note = Full length Pyrococcus sp. 12-1 DNA primase
MRPSDIIIDVYKAIQDHPGAGKLAIELRFYPRPTSEWIIVADIEDKAEELHKVLFKNNVLGKKEAYISMALHDFEEVGKKLEKLRELEEERAQKEGRKPREVTLRNVQGEATGKVHKTVSKYTLTLVVDIDVEEIHKSKVVESEEKAFELAKRAWDELKPKLEGIGVKPRYVFFTGGGVQLWFVAPGLEPIEVIDRASRVIPPVLNAMLPEGYSVDNIFDRARIVRVPLTINYKYKTPDERPLEIRGRLIEFNDVRTPLGEVLDKLEAYAKEHGISLVTPSQARFIGTVGRYEVDKGDFRVLAERLVQELAPWFRRVKEKGGSRHHLVNAIAAYIARNTNLTFEDLAKIWEIVHAELVRLGLEDPNDWSNRYHTIKDVYEKLQAGTFLGTRAYMLKYLNMMSEEEIVEILRAVKRALFPYLKPVNAGFVSKFVDEPYGRDEAPKKWEDVDEDRRKAVGVFFIRALAFETAEYIYVEDLPKPKTFSIKSHSEDGKEKLRFNEALWQSFLSWLGVQEGEPFDRVDFENLLIEKFGITEHELRSIYFSKVLYLLTPEGMRMPKCIVEFLKELADEGNLSDEKVAHLAHWIKYFAKPLRHSTTSIMLRAQGKPVDMRMAVWAKIVEFFAEDDDVAKRLVSVFKKAYEEAEPPFPCFGVKECPFFPGHKGCPFIAPKRNEILAVSLVDVQIHGTDGIVVVVGGGEGTKEFVLRGRVEWVKVSKKGVRYPIAEWFLDVFAKEFLALPEPPAWDPGDVTEILKSRARVVKSQLDEFDEYYEHFIDWLRSENSRGILPYENADSSHLFIKGKMIGIPPRLAEDFYKIDLGISGKKFKEMLIRKLGKHFLGKKPVWIRLTSGEKNGVNCYFVSLEWFRDVVGEPNIKEVEAGEELGEFGFSYEEGEA

SEQ ID NO: 2 - protein, 296 aa, synthetic construct
  REGION 1..296: note = PolpP12297-898
MRPSDIIIDVYKAIQDHPGAGKLAIELRFYPRPTSEWIIVADIEDKAEELHKVLFKNNVLGKKEAYISMALHDFEEVGKKLEKLRELEEERAQKEGRKPREVTLRNVQGEATGKVHKTVSKYTLTLVVDIDVEEIHKSKVVESEEKAFELAKRAWDELKPKLEGIGVKPRYVFFTGGGVQLWFVAPGLEPIEVIDRASRVIPPVLNAMLPEGYSVDNIFDRARIVRVPLTINYKYKTPDERPLEIRGRLIEFNDVRTPLGEVLDKLEAYAKEHGISLVTPSQARFIGTVGRYEVDK

SEQ ID NO: 3 - protein, 290 aa, synthetic construct
  REGION 1..290: note = PolpP1287-92297-898
MRPSDIIIDVYKAIQDHPGAGKLAIELRFYPRPTSEWIIVADIEDKAEELHKVLFKNNVLGKKEAYISMALHDFEEVGKKLEKLREQKEGRKPREVTLRNVQGEATGKVHKTVSKYTLTLVVDIDVEEIHKSKVVESEEKAFELAKRAWDELKPKLEGIGVKPRYVFFTGGGVQLWFVAPGLEPIEVIDRASRVIPPVLNAMLPEGYSVDNIFDRARIVRVPLTINYKYKTPDERPLEIRGRLIEFNDVRTPLGEVLDKLEAYAKEHGISLVTPSQARFIGTVGRYEVDK

SEQ ID NO: 4 - protein, 923 aa, Thermococcus nautili
  REGION 1..923: note = Full length Thermococcus nautili sp. 30-1 DNA primase
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGVNGVKGLLSEGFKADNIFDPARIVRAPLTFNHKYRTIIKDEDGTERVVPTQVKGRVIEFNDVRISLTEFLDRLEAYAKEKGIPLEKPTKRKFLELASKRYEVTSSNFEALAERLFTELRPWWEIAKEKGWSRHHLTMGIATYILRNTNLTPEQLIGSENSPGLWELVFAKLVEAGLEDPDDWKNRASTIRDAYKKIESGKKVATKAYLRKYIEGLSEEDAVQILLSVKRALLPYLKAVDVKRISKYSARPYEITEEIPKSWEDIDENRKKATGVWYIDFLALETANDIYFEDLPKPPVFYIRFVEKNKEKFKLNETLYHSFLTWLGITEGEPLDRTELIDLLVEKFGFTVEDLKAIYYRKILTLLKPEGMRTPKCIQEFLFELATEGNLPEDKIRHLAHWVKFYARPLRHSTTSVLLKGRGKPVDMRLAIWAKVVEFFAEDDEVAEELITTFKKAYMGAEPPFPCIGAESCPFYPDHRACPFIVPKRKEVLPVSIVDVQLHGSDGIVVLVGGPTEVTAFTLEGKVEWIKTTKKTVKYPIAEWFLDRFAKEYLSLPEAPSWWKLEEVTEILKSRARVVKSQFDKFEDYLEQFIEWLQKENSRRGILPYEKADENHLFIKGEWVGIPPGFAREFYSGELLIGGPTFRRMLEQKLGKDYRKMSAKIHLNTGLKDKRNCYFVSVEWFRKHVGEPNIQEITSEGDVSFDGLSYDDEEEGVVG

SEQ ID NO: 5 - protein, 310 aa, synthetic construct
  REGION 1..310: note = PolpTN2311-923
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGVNGVKGLLSEGFKADNIFDPARIVRAPLTFNHKYRTIIKDEDGTERVVPTQVKGRVIEFNDVRISLTEFLDRLEAYAKEKGIPLEKPTKRKFLELASKRYEVTS

SEQ ID NO: 6 - protein, 304 aa, synthetic construct
  REGION 1..304: note = PolpTN290-96311-923
MSSLRPSSIIIDIYKVIQDHPDSSRWAIELRFLPKPISSEWIFITDIEERASEIDKVLTKYNIMKKKDAYVSMAIHDFEKVTAKLKRVQETEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGVNGVKGLLSEGFKADNIFDPARIVRAPLTFNHKYRTIIKDEDGTERVVPTQVKGRVIEFNDVRISLTEFLDRLEAYAKEKGIPLEKPTKRKFLELASKRYEVTS

SEQ ID NO: 7 - protein, 304 aa, synthetic construct
  REGION 1..304: note = PolpTN2205-211311-923
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGLLSEGFKADNIFDPARIVRAPLTFNHKYRTIIKDEDGTERVVPTQVKGRVIEFNDVRISLTEFLDRLEAYAKEKGIPLEKPTKRKFLELASKRYEVTS

SEQ ID NO: 8 - protein, 304 aa, synthetic construct
  REGION 1..304: note = PolpTN2248-254311-923
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGVNGVKGLLSEGFKADNIFDPARIVRAPLTFNHKYRTIIKDEDPTQVKGRVIEFNDVRISLTEFLDRLEAYAKEKGIPLEKPTKRKFLELASKRYEVTS

SEQ ID NO: 9 - protein, 298 aa, synthetic construct
  REGION 1..298: note = PolpTN2243-254311-923
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGVNGVKGLLSEGFKADNIFDPARIVRAPLTFNHKYRTPTQVKGRVIEFNDVRISLTEFLDRLEAYAKEKGIPLEKPTKRKFLELASKRYEVTS

SEQ ID NO: 10 - protein, 298 aa, synthetic construct
  REGION 1..298: note = PolpTN290-96205-211311-923
MSSLRPSSIIIDIYKVIQDHPDSSRWAIELRFLPKPISSEWIFITDIEERASEIDKVLTKYNIMKKKDAYVSMAIHDFEKVTAKLKRVQETEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGLLSEGFKADNIFDPARIVRAPLTFNHKYRTIIKDEDGTERVVPTQVKGRVIEFNDVRISLTEFLDRLEAYAKEKGIPLEKPTKRKFLELASKRYEVTS

SEQ ID NO: 11 - protein, 298 aa, synthetic construct
  REGION 1..298: note = PolpTN290-96248-254311-923
MSSLRPSSIIIDIYKVIQDHPDSSRWAIELRFLPKPISSEWIFITDIEERASEIDKVLTKYNIMKKKDAYVSMAIHDFEKVTAKLKRVQETEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGVNGVKGLLSEGFKADNIFDPARIVRAPLTFNHKYRTIIKDEDPTQVKGRVIEFNDVRISLTEFLDRLEAYAKEKGIPLEKPTKRKFLELASKRYEVTS

SEQ ID NO: 12 - protein, 292 aa, synthetic construct
  REGION 1..292: note = PolpTN290-96243-254311-923
MSSLRPSSIIIDIYKVIQDHPDSSRWAIELRFLPKPISSEWIFITDIEERASEIDKVLTKYNIMKKKDAYVSMAIHDFEKVTAKLKRVQETEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGVNGVKGLLSEGFKADNIFDPARIVRAPLTFNHKYRTPTQVKGRVIEFNDVRISLTEFLDRLEAYAKEKGIPLEKPTKRKFLELASKRYEVTS

SEQ ID NO: 13 - protein, 298 aa, synthetic construct
  REGION 1..298: note = PolpTN2205-211248-254311-923
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGLLSEGFKADNIFDPARIVRAPLTFNHKYRTIIKDEDPTQVKGRVIEFNDVRISLTEFLDRLEAYAKEKGIPLEKPTKRKFLELASKRYEVTS

SEQ ID NO: 14 - protein, 292 aa, synthetic construct
  REGION 1..292: note = PolpTN2205-211243-254311-923
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGLLSEGFKADNIFDPARIVRAPLTFNHKYRTPTQVKGRVIEFNDVRISLTEFLDRLEAYAKEKGIPLEKPTKRKFLELASKRYEVTS

SEQ ID NO: 15 - protein, 292 aa, synthetic construct
  REGION 1..292: note = PolpTN290-96205-211248-254311-923
MSSLRPSSIIIDIYKVIQDHPDSSRWAIELRFLPKPISSEWIFITDIEERASEIDKVLTKYNIMKKKDAYVSMAIHDFEKVTAKLKRVQETEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGLLSEGFKADNIFDPARIVRAPLTFNHKYRTIIKDEDPTQVKGRVIEFNDVRISLTEFLDRLEAYAKEKGIPLEKPTKRKFLELASKRYEVTS

SEQ ID NO: 16 - protein, 286 aa, synthetic construct
  REGION 1..286: note = PolpTN290-96205-211243-254311-923
MSSLRPSSIIIDIYKVIQDHPDSSRWAIELRFLPKPISSEWIFITDIEERASEIDKVLTKYNIMKKKDAYVSMAIHDFEKVTAKLKRVQETEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGLLSEGFKADNIFDPARIVRAPLTFNHKYRTPTQVKGRVIEFNDVRISLTEFLDRLEAYAKEKGIPLEKPTKRKFLELASKRYEVTS

SEQ ID NO: 17 - protein, 928 aa, Thermococcus sp.
  REGION 1..928: note = Full length Thermococcus sp. CIR10 DNA primase
MSGREFKRPSDVIIDIYKVIQDHPEAGRLAIEFRFYPYPTSEWILLNDIEDKAREIDKVLFKNNILGKKEAYISMAIHDFDEVTKKLEKLQELEHEKAQKEGRQPKEITLRHVQGEATGKIHTTVSSYTLTLVVDIDVNEIHDSKAVESEEKALEVSKRAWEVLKPNLEELGIKPRYVFFTGGGIQLWFVAPEPENISVIDKAAEIIPPVLNTLLPEGYSVDNIFDRARIVRVPFTVNYKYKTPDGKPLELRGRLLEFNDVRTPLGDILEKLEAYAKGHKISLGSTSRSGKFRGVAGRYEVKKENFEELAKRLVEELAPWFKKIKERGGSRHHLVNAIAAYIARNTNLTEEDLLGKDQEDGTHVVGLWELVHSKLVELGLEDPGDWSNRYHTIKDVYEKLYAGTTLGTRAYMMKYLNVSEEEAIEILRSVKRALFPYLHPVNVQVISKFEAKPYSKEEAPTEWEAVDEDRKKAVGIWYIEVLALETANYVYIEDLSKPGVFYIVEKVKRTVKVGKKEKGVEVDEYHFNPALWQSFLNWLGIKEGEPIEREELWNLLLEKFDIKDYELRAIYFRKILHLLSPEGMRRPRCVEEFLRELADEGFLSEDKVRHLAHWIKFYAKPLRHSTTSIMLRAKGKPVDMRMAVWAKVVEFFAEDEETAQGLIETFREAYEQAEPPFPCFGARECPFFQEHRGCPFIAPKRDEILAVSLVDVQLHGSDGIVIIVGSEEGTKKFVHKGKVEWQKQGKSKIKYPVAEWFLDVYAKEFLSLPEAPSWSHEEVTEILKSRARVVRSQLNEFDEYFDNFIDWLRSENARGIYPYEKADSSHIFIKGNMIGIPPRLAEDFYRNELGISGRKFKEMLIRELGSYYLGKKAAWIKLSSGQHNGVNCYFISLDWFKKIVGEPNIKDIEAEGDIGSGGFNYEEEEGEA

SEQ ID NO: 18 - protein, 303 aa, synthetic construct
  REGION 1..303: note = PolpCIR10303-928
MSGREFKRPSDVIIDIYKVIQDHPEAGRLAIEFRFYPYPTSEWILLNDIEDKAREIDKVLFKNNILGKKEAYISMAIHDFDEVTKKLEKLQELEHEKAQKEGRQPKEITLRHVQGEATGKIHTTVSSYTLTLVVDIDVNEIHDSKAVESEEKALEVSKRAWEVLKPNLEELGIKPRYVFFTGGGIQLWFVAPEPENISVIDKAAEIIPPVLNTLLPEGYSVDNIFDRARIVRVPFTVNYKYKTPDGKPLELRGRLLEFNDVRTPLGDILEKLEAYAKGHKISLGSTSRSGKFRGVAGRYEVKK

SEQ ID NO: 19 - protein, 297 aa, synthetic construct
  REGION 1..297: note = PolpCIR1093-98303-928
MSGREFKRPSDVIIDIYKVIQDHPEAGRLAIEFRFYPYPTSEWILLNDIEDKAREIDKVLFKNNILGKKEAYISMAIHDFDEVTKKLEKLQEQKEGRQPKEITLRHVQGEATGKIHTTVSSYTLTLVVDIDVNEIHDSKAVESEEKALEVSKRAWEVLKPNLEELGIKPRYVFFTGGGIQLWFVAPEPENISVIDKAAEIIPPVLNTLLPEGYSVDNIFDRARIVRVPFTVNYKYKTPDGKPLELRGRLLEFNDVRTPLGDILEKLEAYAKGHKISLGSTSRSGKFRGVAGRYEVKK

SEQ ID NO: 20 - protein, 914 aa, Thermococcus peptonophilus
  REGION 1..914: note = Full length Thermococcus peptonophilus DNA primase
MSELTPGKVLADVYKVIQDHPEAGRLAIELRFYPVIKSEWVLLNDIEDKARDIDKVLAKQNLIKGKEAYVSMAIHGFEAVKKKLEKLRESVEEGKVRKLGLENVQGEAKGKVHPTVSNYTLTLVVDVDIEAVHKLKVVEDVDKVFEKAKEGWLALKPVFEELGVLPRYVFFTGGGLQLWFVAPKLEDIAVIDRASGIVPNVLNALLPEGFVVDNIFDRARIVRAPLTVNHKYKAPNGARVGVKGRLIEFNDVRVSLSEVLDKLEVYAKERGIQLGGQEKVRGGRGFVNVRYVVKKEELETLALNLADELIPWFKKVKERGGSWHHLVNAIGAYVVRNTNLSLEDLIGKDNPDGTHVVGLWEIVFQRLVEKSAEDPGDWVNRRNTIKDVYEKHIAGKPLGTRAYLKKYLPVSDEEVVEILMAVRRALLPFLKEVKKVDSSGFGVAPYKKTAPRSWDEVDEDRKRATGRWYVWKLAFNTAEYLFTDELPKAGTFYIDVWMKEGKREWMKRFFNEALFRSFIEDGLGYKYGAPVEREELFERLVEVFNITDEEVRGIYIDAALSLLSPVGMRTPPCIEEFIMEFAANGSLSEDKVRHLARWIKLYAKPLKHSTTTTKLVGAGYKVDMRMAVWAKLVEFFAEDDEVARELVRVFKEEYGAAEPPFTCIGTKTCQFYLNEKMCPFIIPKEKEILAVSLIDVQRHESDGLVVIVGGDKEVRTFVKKGNVEWVKKTERREKYPVAEWFIDVFATEYLSVSPDDLDVDLEEVTDILKSRARVVKSRLNELEDMYEKFVEWLKRENAVRGVLPYEKADFNHLFIKGNMIGIPPALAEEFYRFELNIKGSEFREMLEKKLGFHYTKKAVKLSVGEKKDVRRCYLVSLEWFRKVVGEPNVKDVVMAGDIALSGLVYYESGEEVVE

SEQ ID NO: 21 - protein, 295 aa, synthetic construct
  REGION 1..295: note = PolpTpep295-914
MSELTPGKVLADVYKVIQDHPEAGRLAIELRFYPVIKSEWVLLNDIEDKARDIDKVLAKQNLIKGKEAYVSMAIHGFEAVKKKLEKLRESVEEGKVRKLGLENVQGEAKGKVHPTVSNYTLTLVVDVDIEAVHKLKVVEDVDKVFEKAKEGWLALKPVFEELGVLPRYVFFTGGGLQLWFVAPKLEDIAVIDRASGIVPNVLNALLPEGFVVDNIFDRARIVRAPLTVNHKYKAPNGARVGVKGRLIEFNDVRVSLSEVLDKLEVYAKERGIQLGGQEKVRGGRGFVNVRYVVKK

SEQ ID NO: 22 - protein, 913 aa, Thermococcus celericrescens
  REGION 1..913: note = Full length Thermococcus celericrescens DNA primase
MSELTPGKVLADVYKVIQDHPEAGRLAIELRFYPVIKSEWVLLNDIEDKARDIDKVLAKKNIINGKEAYVSMAIHDFGAVKKKLEKLREKAEGERARRIGLENVQGEAKGKVHPTVSNYTLALVVDIDIEEVHKSRVVEDVEAVFERAKKGWLALRPVFEELGVLPRYVFFTGGGLQIWFVAPELEDIAVIDRASGIVPGVLNALLPEGFVVDNIFDRARIVRAPLTVNHKYKAPNGAGLGVKGRLIEFNDVRVSLSEVLDKLEAYAKEKGIQLGGQERASGVRVFGKVRYEVKKERLETLALNLADELAPWFKKVKERGGSWHHLVNAIGAYIVRNTNLSLEDLIGKDNPDGTHVVGLWELVFQRLVEKGAEDPSDWLNRRNTIKDVYEKHIAGKPLGTRAYLKKYLPVSDEEVVEILMAARRALLPFLKGVKRAGSSGFGVFPYKDTAPRSWNEVEAERKRATGLWYVWSLAFSTAEYIFTDELSKAGTFFILVKEGKSIKSVFNETLFRSFIEEGLGYKYGMPVRRDELFERLVEVFNITDEEVRGVYIDAALSLLSPVGMRTPPCIEEFIMEFAANGNLPEDKVRHLARWIKLYAKPLKHSTTTTKLVGAGYNVDMRMAVWAKLVEFFAEDDEVAGELVRIFKEEYKEAEPPFTCIGARTCPFYLKDDVAKMCPFIFPKEKEILAVSLIDVQRHESDGIVILVGGAREVRTFIKKGKVEWVKRTKKTEKYPIAEWFIDVFATEYLGVPPDNLDFDLKDVTGILKSRERVVPSQLNEVEDWYEKFVEWLKRENEVRGVLPYKKADVHHLFIKDNMIGIPPALAEEFYRYEANIKPSEFREMLEVKLSIHYKKKAVKLSVGEKKDVRRCYLVSLEWFRKVVGEPNVKDVVMAGDIALSGLVYYESGEEVVE

SEQ ID NO: 23 - protein, 295 aa, synthetic construct
  REGION 1..295: note = PolpTcele295-913
MSELTPGKVLADVYKVIQDHPEAGRLAIELRFYPVIKSEWVLLNDIEDKARDIDKVLAKKNIINGKEAYVSMAIHDFGAVKKKLEKLREKAEGERARRIGLENVQGEAKGKVHPTVSNYTLALVVDIDIEEVHKSRVVEDVEAVFERAKKGWLALRPVFEELGVLPRYVFFTGGGLQIWFVAPELEDIAVIDRASGIVPGVLNALLPEGFVVDNIFDRARIVRAPLTVNHKYKAPNGAGLGVKGRLIEFNDVRVSLSEVLDKLEAYAKEKGIQLGGQERASGVRVFGKVRYEVKK

SEQ ID NO: 24 - protein, 296 aa, synthetic construct
  REGION 1..296: note = SHEE 4.3/13.1/3.24
MRPSDIIIDVYKAIQDHPGAGKLAIELRFYPRPTSEWIIVADIEDKAEELHKVLFKNNVLGKKEAYISMALHDFEEVGKKLEKLRELEEERAQKEGRKPREVTLRNVQGEATGKVHKTVSKYTLTLVVDIDVEEIHKSKVVESEEKAFELAKRAWDELKPKLEGIGVKPRYVFFTGGGVQLWFVAPGLEPIEVIDRASRVIPPVLRAMLPEGYSVDKIFDRARIVRVPLTINHKYKTPDERPLEIRGRLIEFNDVRTPLGEVLDKLEAYAKEHGISLVTPSQARFIGTVGRYEVDK

SEQ ID NO: 25 - protein, 296 aa, synthetic construct
  REGION 1..296: note = SHEE 4.3/13.1/9.2
MRPSDIIIDVYKAIQDHPGAGKLAIELRFYPRPTSEWIIVADIEDKAEELHKVLFKNNVLGKKEAYISMALHDFEEVGKKLEKLRELEEERAQKEGRKPREVTLRNVQGEATGKVHKTVSKHTLTLVVDIDVEEIHKSKVVESEEKAFELAKRAWDELKPKLEGIGVKPRYVFFTGGGVQLWFVAPGLEPIEVIDRASRVIPPVLRAMLPEGYSVDKIFDRARIVRVPLTINYKYKTPDERPLEIRGRLIEFNDVRTPLGEVLDKLEAYAKEHGISLVTPSQARFIGTVGRYEVDK

SEQ ID NO: 26 - protein, 296 aa, synthetic construct
  REGION 1..296: note = SHEE 4.3/13.1/11.1
MRPSDIIIDVYKAIQDHPGAGKLAIELRFYPRPTSEWIIVADIEDKAEELHKVLFKNNVLGKKEAYISMALHDFEEVGKKLEKLRELEEERAQKEGRKPREVTLRNVQGEATGKVHKTVSKYTLTLVVDIDVEEIHKSKVVESEEKAFELAKRAWDELKPKLEGIGVKPRYVFFTGGGVQLWFVAPGLEPIEVIDRASRVIPPVLRAMLPEGYSVDKIFDRARIVRVPLTINYRYKTPDERPLEIRGRLIEFNDVRTPLGEVLDKLEAYAKEHGISLVTPSQARFIGTVGRYEVDK

SEQ ID NO: 27 - protein, 142 aa, Thermotoga neapolitana
  REGION 1..142: note = Single stranded DNA-binding protein (ssb)
MSFFNRIILIGRLVRDPEERYTLSGTPVTTFTIAVDRVPRKNAPDDAQTTDFFRVVTFGRLAEFARTYLTKGRLILVEGEMRMRRWETQTGEKRVSPEVVANVVRFMDRKPVEMPSEDIEEKLEIPEEDFTDDTFSEDEPPF

SEQ ID NO: 28 - protein, 449 aa, synthetic construct
  REGION 1..449: note = SHEE-N18
MSFFNRIILIGRLVRDPEERYTLSGTPVTTFTIAVDRVPRKNAPDDAQTTDFFRVVTFGRLAEFARTYLTKGRLILVEGEMRMRRWETQTGEKRVSPEVVANVVRFMDRKPVEMPSEDIEEKLEIPEEDFTDDTFSEDEPPFRWGSELEMRPSDIIIDVYKAIQDHPGAGKLAIELRFYPRPTSEWIIVADIEDKAEELHKVLFKNNVLGKKEAYISMALHDFEEVGKKLEKLRELEEERAQKEGRKPREVTLRNVQGEATGKVHKTVSKYTLTLVVDIDVEEIHKSKVVESEEKAFELAKRAWDELKPKLEGIGVKPRYVFFTGGGVQLWFVAPGLEPIEVIDRASRVIPPVLNAMLPEGYSVDNIFDRARIVRVPLTINYKYKTPDERPLEIRGRLIEFNDVRTPLGEVLDKLEAYAKEHGISLVTPSQARFIGTVGRYEVDKGTGT

SEQ ID NO: 29 - protein, 444 aa, synthetic construct
  REGION 1..444: note = SHEE-C18
MRPSDIIIDVYKAIQDHPGAGKLAIELRFYPRPTSEWIIVADIEDKAEELHKVLFKNNVLGKKEAYISMALHDFEEVGKKLEKLRELEEERAQKEGRKPREVTLRNVQGEATGKVHKTVSKYTLTLVVDIDVEEIHKSKVVESEEKAFELAKRAWDELKPKLEGIGVKPRYVFFTGGGVQLWFVAPGLEPIEVIDRASRVIPPVLNAMLPEGYSVDNIFDRARIVRVPLTINYKYKTPDERPLEIRGRLIEFNDVRTPLGEVLDKLEAYAKEHGISLVTPSQARFIGTVGRYEVDKGTMEFEASFFNRIILIGRLVRDPEERYTLSGTPVTTFTIAVDRVPRKNAPDDAQTTDFFRVVTFGRLAEFARTYLTKGRLILVEGEMRMRRWETQTGEKRVSPEVVANVVRFMDRKPVEMPSEDIEEKLEIPEEDFTDDTFSEDEPPF

SEQ ID NO: 30 - protein, 310 aa, synthetic construct
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGHTLALVVDIDNTKIHDTRIIENEEEAFEASKREWEALKPKLQELGFLPRWILYTGGGLQLWFVSDKLEPISVIDRASEIIPNIMRGVNGVKGLLSEGFKADKIFDPARIVRAPLTFNHKYRTIIKDEDGTERVVPTQVKGRVIEFNDVRISLTEFLDRLEAYAKEKGIPLEKPTKRKFLELASKRYEVTS

SEQ ID NO: 31 - protein, 310 aa, synthetic construct
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMRGVNGVKGLLSEGFKADKIFDPARIVRAPLTFNHRYRTIIKDEDGTERVVPTQVKGRVIEFNDVRISLTEFLDRLEAYAKEKGIPLEKPTKRKFLELASKRYEVTS

SEQ ID NO: 32 - protein, 310 aa, synthetic construct
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGHTLALVVDIDNTKIHDTRIIENEEEAFEASKREWEALKPKLQELGFLPRWILYTGGGLQLWFVSDKLEPISVIDRASEIIPNIMRGVNGVKGLLSEGFKADKIFDPARIVRAPLTFNHRYRTIIKDEDGTERVVPTQVKGRVIEFNDVRISLTEFLDRLEAYAKEKGIPLEKPTKRKFLELASKRYEVTS